(19) United States
(12) Patent Application Publication
(10) Pub. No.: US 2011/0094531 A1
(43) Pub. Date: Apr. 28, 2011

(54) COMPOSITION FOR TREATING KERATIN FIBERS, COMPRISING AT LEAST ONE POLYALKENE-BASED SUPRAMOLEDCULAR POLYMER, AT LEAST ONE BLOCK POLYMER, AND AT LEAST ONE VOLATILE SOLVENT

(76) Inventor: Karima ABBAS, Saint Denis (FR)

(21) Appl. No.: 12/818,411

(22) Filed: Jun. 18, 2010

Related U.S. Application Data
(60) Provisional application No. 61/226,105, filed on Jul. 16, 2009.

(30) Foreign Application Priority Data
Jun. 18, 2009 (FR) 0954105

(51) Int. Cl. A61K 8/90; A61Q 5/00; A61Q 5/06

(52) U.S. Cl. 132/202; 424/70.16; 424/70.12; 424/70.121; 424/70.6

(57) ABSTRACT

Disclosed herein is a composition for treating keratin fibers, comprising: at least one polyalkene-based supramolecular polymer, at least one block ethylenic copolymer, and at least one volatile solvent. Also disclosed is a process for treating keratin fibers comprising applying to the keratin fibers the composition for treating keratin fibers.

This application claims benefit of U.S. Provisional Application No. 61/226,105, filed Jul. 16, 2009. This application also claims benefit of priority under 35 U.S.C. §119 to French Patent Application No. 0954105, filed Jun. 18, 2009.

Disclosed herein is a composition for treating keratin fibers, and for example human keratin fibers such as the hair, and also a composition for dyeing the hair using a pigment.

The hair can be damaged and embrittled by the action of external atmospheric agents such as light and bad weather, and/or by mechanical or chemical treatments such as brushing, combing, bleaching, permanent-waving and/or dyeing. As a result, the hair can be often difficult to manage, for example, to disentangle and style, and a head of hair, even a lush head of hair, can have difficulty holding an attractive hairstyle due to the fact that the hair lacks vigour, volume and liveliness.

This degradation of the hair can be, moreover, accentuated by repeated permanent hair-dyeing treatments, which comprise applying to the hair a dye precursor and an oxidizing agent.

Thus, in order to remedy this, it may be now common to use styling products which make it possible to condition the hair, giving the latter certain body, bulk or volume.

These styling products can be cosmetic hair compositions comprising at least one polymer which may have a strong affinity for the hair and which may have the function of forming a film at the surface of the hair for the purpose of modifying the surface properties of said hair, such as conditioning said hair.

One drawback that can be linked to the use of these hair compositions lies in the fact that the cosmetic effects conferred by such compositions have a tendency to disappear, for example, as soon as the hair is shampooed for the first time.

It can be known from patent EP 1 392 222, to use a cosmetic composition for caring for and/or treating keratin materials, comprising a supramolecular polymer comprising a polymeric backbone and at least two groups capable of forming at least three hydrogen bonds, and from patent EP 1 435 900, to use a hair composition comprising a supramolecular polymer comprising a polymeric backbone and at least two groups capable of forming at least three hydrogen bonds and a surfactant or a hair conditioning agent, in order to improve the persistence of the coatings.

For example, coatings can be obtained using a polyalkene-based supramolecular polymer. When these polymers are used on the hair, the hairs may be coated homogeneously while at the same time remaining individualized. This coating may give the head of hair body and bulk which persist. When the above coating contains pigments, the coloring obtained can be chromatic and highly visible on a dark background.

One drawback that may be linked to coatings using a polyalkene-based supramolecular polymer may be their lack of resistance to external attacks such as fatty substances and, for example, sebum. The sensitivity of the coating to fatty substances can result in a decrease in the resistance of the coating to shampooing and in a degradation of its aesthetics, the hair becoming sticky to the touch.

Thus, disclosed herein is a composition for treating keratin fibers, and for example the hair, which makes it possible to obtain a coating of the hair that can be persistent with respect to shampooing, with the improved properties of resistance to external attacks, such as sebum, without degradation of the keratin fibers and while at the same time conserving hairs which can be individualized and non-sticky.

Thus, provided herein is a composition for treating keratin fibers, and for example the hair, comprising:

at least one polyalkene-based supramolecular polymer,

at least one block ethylenic copolymer comprising

at least one first block having a glass transition temperature (Tg) of greater than or equal to 40° C. which comprises at least one first monomer whose corresponding homopolymer has a glass transition temperature of greater than or equal to 40° C., and

at least one second block having a glass transition temperature of less than or equal to 20° C. which comprises at least one second monomer, whose corresponding homopolymer has a glass transition temperature of less than or equal to 20° C.,

wherein the at least one first block and the at least one second block are linked to one another via a random intermediate segment comprising at least one constituent monomer of the at least one first block and at least one constituent monomer of the at least one second block,

and wherein the at least one block copolymer has a polydispersity index I of greater than 2, and

at least one volatile solvent.

The composition in accordance with the present disclosure makes it possible to obtain, on the keratin fibers, coatings that can be persistent with respect to shampooing and which can preserve the physical qualities of the keratin fiber. Such a coating can be for example resistant to the external attacks to which the hair may be subjected, such as blow-drying and perspiration, further such as fatty substances such as sebum. It makes it possible for example to obtain a smooth and homogeneous deposit with individualized hairs, that can be styled without difficulty.

The term “individualized hairs” is intended to mean hairs which, after application of the composition and drying, are not stuck together (or are all separate from one another) and therefore do not form clumps of hair, since the coating can be formed around virtually every hair.

Provided herein is also a process for treating keratin fibers, and for example the hair, using this composition.

### Polyalkene-Based Supramolecular Polymers

As disclosed herein, the term “polyalkene-based supramolecular polymer” is intended to mean a polymer comprising, in its structure, at least one polyalkene part and at least one part comprising at least one group capable of forming at least three H-bonds, such as four H-bonds.

The polyalkene is for example chosen from poly(ethylene-butylene)s, polybutadienes and polyisoprenes.

The supramolecular polymers may be derived from the condensation of at least one polyalkene polymer functionalized with at least one reactive group, together with at least one graft functionalized with at least one reactive group capable of reacting with the at least one reactive group of the functionalized polyalkene, wherein said graft comprises at least one group capable of forming at least three H-bonds, for example at least four H-bonds.

For example, the functionalized polyalkene polymer can be of formula (A):

HX—R—X′H (A)

XH and X′H are reactive groups, with X and X′, which may be identical or different, chosen from O, S, NH and NRₐ, wherein Rₐ represents a C₁-C₆ alkyl group; for example, X and/or X′ denote O; for further example, X and X′ denote O;

R represents a homopolymer or a copolymer comprising at least one monounsaturated or polyunsaturated C₂-C₁₀, and for example C₂-C₄, alkenes; R, for example, represents a poly(ethylene-butylene), a polybutadiene or a polyisoprene.

The poly(ethylene-butylene)s are copolymers of 1-butylene and of ethylene. They can be represented schematically by the series of units of structures:

[—CH₂—CH₂—] and [—CH₂CH(CH₂—CH₃)].

The polybutadienes may be 1,4-polybutadienes or 1,2-polybutadienes, which can be respectively represented schematically by the series of units below:

[—CH₂—CH═CH—CH₂—] (1,4-polybutadienes)

[—CH₂—CH(CH═CH₂)—] (1,2-polybutadienes)

According to at least one embodiment, the polybutadienes are 1,2-polybutadienes.

The functionalization can be, for example carried out at the end of the chains. The term “telechelic polymers” is then used. The functionalizing groups can be attached to the polyalkene via linkers, such as linear or branched C₁-C₄ alkylene groups.

These polyalkenes may be partially or totally hydrogenated so as to limit or avoid the risks of crosslinking.

They may comprise, in their structure, other monomers. As comonomers, exemplary mention may be made of styrene.

Hydroxyl-terminated polydienes, which are for example hydrogenated, and hydroxyl-terminated polyolefins are the polymeric backbones according to at least one embodiment of the disclosure.

These hydroxyl-terminated polydienes may be defined, for example, in patent FR 2 782 723. They can be chosen from homopolymers and copolymers of polybutadiene, of polyisoprene and of poly(1,3-pentadiene). They can be oligomers with a number-average molecular mass of less than 7,000, and for example from 1,000 to 5,000, having a terminal hydroxyl functionality of 1.8 to 3, and for example in the region of 2.

Exemplary mention may be made of the hydroxylated polybutadienes sold by the company Elf Atochem under the trademarks POLY BD R-45HT and POLY BD R-20 LM, which will for example be used hydrogenated. Mention may also be made of di-OH hydrogenated (1,2-polybutadiene)s, such as the GI3000 of Mn=2600-3200 and the GI2000 of Mn=1800-2200 sold by the company Nisso.

Use may also be made of α,ω-hydroxyl-terminated polyolefins, homopolymers or copolymers, such as:

α,ω-hydroxyl-terminated polyisobutylene oligomers;

the copolymers sold by the company Mitsubishi under the trademark POLYTAIL with, for example, those corresponding to the formula:

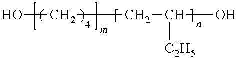

The supramolecular polymers of the present disclosure have, in their structure, at least one graft which comprises at least one group capable of forming at least three H-bonds, such as at least four H-bonds.

These groups capable of forming at least three H-bonds may comprise, for example, at least three functional groups, such as at least four, chosen from:

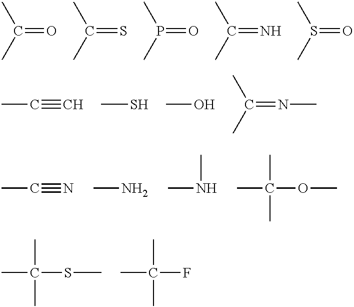

These functional groups may be classified in two categories:

H-bond-donor functional groups such as the groups:

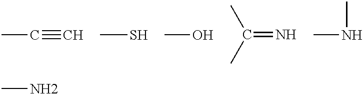

and H-bond-acceptor functional groups such as the groups:

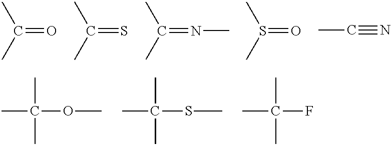

The groups capable of forming at least three H-bonds form a basic structural element comprising at least three functional groups, for example at least four functional groups capable of establishing H-bonds. The basic structural elements capable of establishing three or four H-bonds may be represented schematically in the following way:

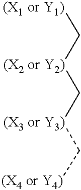

where Xᵢ (i=natural integer) is an H-bond-acceptor functional group and Yᵢ is an H-bond-donor functional group.

Thus, each structural element should be able to establish H-bonds with at least one partner structural element, which may be identical (i.e. self-complementary) or different, such that each pairing of two partner structural elements takes place by formation of at least three H-bonds, such as at least four H-bonds.

An H-bond-acceptor X will pair with an H-bond-donor Y.

Several possibilities are offered, for example pairing of:

XXXX with YYYY;

XXXY with YYYX;

XXYX with YYXY;

XYYX with YXXY;

XXYY with YYXX, self-complementary or not;

XYXY with YXYX, self-complementary or not.

According to at least one embodiment, the groups can establish four H-bonds with an identical (or self-complementary) partner group, among which bonds are two donor bonds (for example, NH) and two acceptor bonds (for example, CO and —C═N—).

According to at least one embodiment, the groups capable of forming at least three H-bonds comprise rings with 5 or 6 atoms (for example, unsaturated heterocycles or aromatic rings), which can be constituted of C and/or N atoms, and with conjugated double bonds in order to stabilize and direct the H interactions.

According to at least one embodiment, the groups capable of forming at least three H-bonds comprise rings with 6 atoms comprising C and/or N atoms and with conjugated double bonds in order to stabilize and direct the H interactions.

According to at least one embodiment, the groups capable of forming three or four H-bonds are chosen from the following compounds, including the tautomeric forms thereof:

(i) the aminopyrimidones of formula:

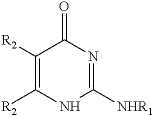

(ii) the ureidopyrimidones of formula:

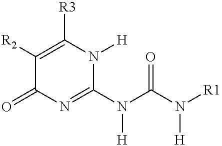

(iii) acylaminopyridines, and for example:

the monoacylaminopyridines of structure:

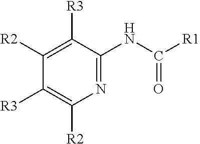

di(acylamino)pyridines, and for further example the 2,6-di(acylamino)pyridines of structure:

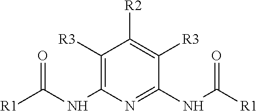

(iv) aminopyrimidines, and for example:

the aminopyrimidine compounds:

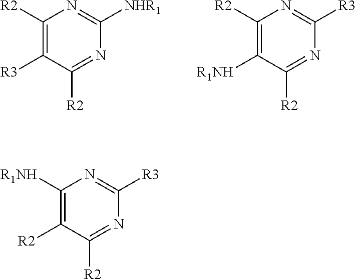

the diaminopyrimidine compounds of structure:

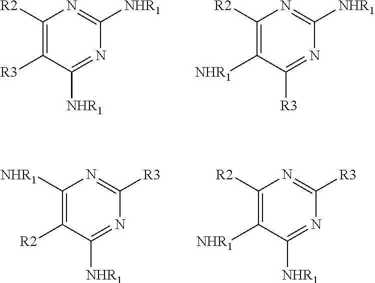

triaminopyrimidine compounds;

(v) ureidotriazines, and for example mono-, di- and triureidotriazines, and for further example the ureidoaminotriazines of structure:

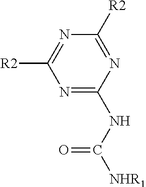

(vi) (acylamino)triazines, and for example mono-, di- and tri(acylamino)triazines, optionally amino (mono-, di- or triamino), and for further example:

di(acylamino)triazines of structure:

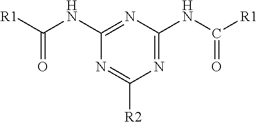

(acylamino)aminotriazines (mono- or di(acylamino), and mono- or diamino), and for example the compounds of structure:

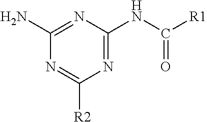

(acylamino)triazines of structure:

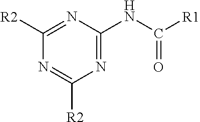

tri(acylamino)triazines;

(vii) aminotriazines, and for example:

monoaminotriazines;

2,6-diamino-s-triazines of structure:

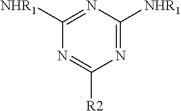

triamino-s-triazine compounds of structure:

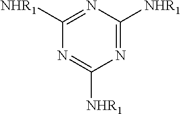

(viii) acylaminotriazoles of structure:

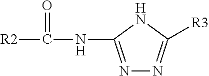

(ix) the compounds of the urazoylbenzoic acid family of structure:

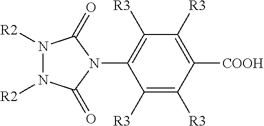

(x) phthalhydrazides of structure:

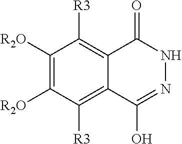

(xi) uracils of structure:

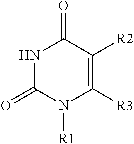

(xii) thymines of structure:

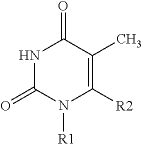

(xiii) succinimides of structure:

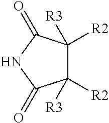

(xiv) glutarimides of structure:

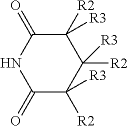

(xv) the compounds of the cyanuric acid family of structure:

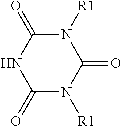

(xvi) maleimides:

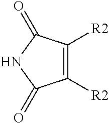

(xvii) the compounds of the barbituric acid family, of structure:

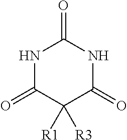

(xviii) the compounds of structure:

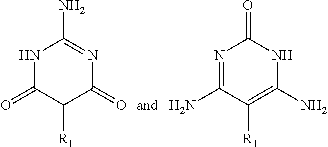

(xix) the compounds of the trimellitic acid family, of formula:

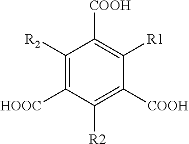

(xx) ureidopyridines, for example mono- or diureidopyridines, and for further example those of the formula:

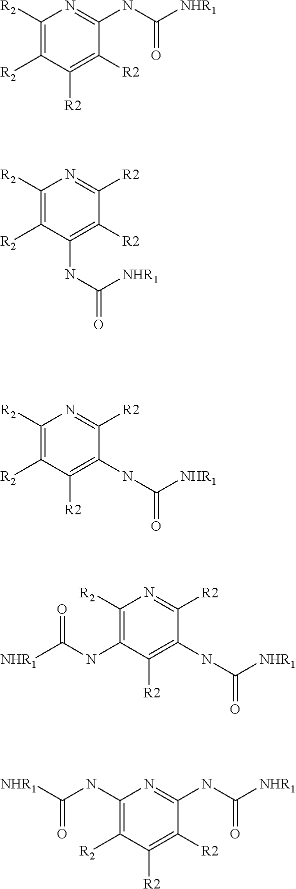

(xxi) carbamoylpyridines, of formula:

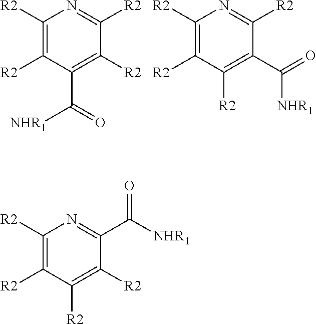

(xxii) adenines of formula:

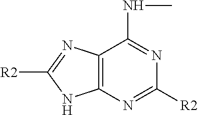

(xxiii) guanines of formula:

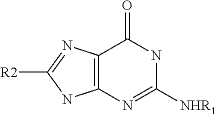

and

(xxiv) cytidines of formula:

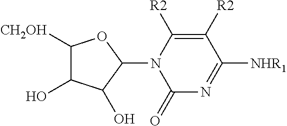

wherein:

(a) the R₁ radicals, which may be identical or different, represent a single bond, a hydrogen atom, a halogen atom, and/or a saturated or unsaturated, optionally aromatic, linear, branched or cyclic monovalent C₁-C₆₀₀₀ carbon-based (for example alkyl) group that may comprise at least one heteroatom such as O, S, N, P, Cl, Br or F;

The R₁ radical may for example be a C₄-C₁₂ cycloalkyl group, a linear or branched C₁-C₃₀ alkyl group or a C₄-C₁₂ aryl group, optionally substituted with at least one group chosen from an amino, ester and hydroxyl.

The R₁ radical may also be chosen from: C₄H₉; phenyl; 1,4-nitrophenyl; 1,2-ethylene; 1,6-hexylene; 1,4-butylene; 1,6-(2,4,4-trimethylhexylene); 1,4-(4-methylpentylene); 1,5-(5-methylhexylene); 1,6-(6-methylheptylene); 1,5-(2,2,5-trimethylhexylene); 1,7-(3,7-dimethyloctylene); -isophorone-; 4,4′-methylenebiscyclohexylene; tolylene; 2-methyl-1,3-phenylene; 4-methyl-1,3-phenylene; and 4,4-biphenylenemethylene;

and for example chosen from: -isophorone-; —(CH₂)₂—; —(CH₂)₆—; —CH₂CH(CH₃)—CH₂—C(CH₃)₂—CH₂—CH₂; 4,4′-methylenebiscyclohexylene; and 2-methyl-1,3-phenylene.

According to at least one embodiment, R₁ is a single bond;

(b) the R₂ radicals, which may be identical or different within the same formula, represent a single bond, a hydrogen atom, a halogen atom (for example, —Br, —Cl, and —F), an —OH or —N(R)₂, (with R being H or a linear or branched C₁-C₁₂, such as C₁-C₄ alkyl radical, and further such as a methyl or ethyl), or a saturated or unsaturated, optionally aromatic, linear, branched or cyclic monovalent C₁-C₆₀₀₀ hydrocarbon-based group that may comprise at least one heteroatom such as O, S, N, P or F;

The R₂ radicals may for example be H, CN, NH₂ and/or:

a C₁-C₃₀ alkyl group;

a C₄-C₁₂ cycloalkyl group;

a C₄-C₁₂ aryl group;

a (C₄-C₁₂)aryl(C₁-C₃₀)alkyl group;

a C₁-C₄ alkoxy group;

an arylalkoxy group, such as a (C₁-C₄)arylalkoxy group;

a C₄-C₁₂ heterocycle;

a thioalkoxy group;

a sulphoxy group;

wherein these groups can be optionally substituted with at least one group chosen from an amino, ester and hydroxyl.

According to at least one embodiment, R₂ represents H, CH₃, C₁₃H₂₇, C₇H₁₅ or phenyl;

(c) the R₃ radicals, which may be identical or different within the same formula, represent a hydrogen atom or a saturated or unsaturated, optionally aromatic, linear, branched or cyclic monovalent C₁-C₆₀₀₀ hydrocarbon-based group that may comprise at least one heteroatom such as O, S, N, P or F;

The R₃ radical may for example be a C₄-C₁₂ cycloalkyl group, a linear or branched C₁-C₃₀ alkyl group or a C₄-C₁₂ aryl group, optionally substituted with at least one group chosen from an amino, ester and hydroxyl. According to at least one embodiment, the R₃ radical represents a methyl radical.

Provided that in all of these formulae, at least one, for example one or two, of the R₁ and/or R₂ group is a single bond constituting the point of attachment of the group capable of forming at least three H-bonds on the residue of the graft.

According to at least one embodiment, said point of attachment is carried by R₁ and/or R₂, and it is for example carried by R₁.

The groups capable of forming at least three H-bonds may for example be chosen from:

(a) groups capable of forming at least three H-bonds which are complementary and identical, e.g. self-complementary, and for example:

aminopyrimidones, ureidopyrimidones,

compounds of the trimellitic acid family and of the urazoylbenzoic acid family,

acylaminopyridines, ureidopyridines, carbamoylpyridines,

acylaminotriazines, ureidotriazines, and for example ureidoaminotriazines, diaminotriazines,

acylaminotriazoles,

phthalhydrazides, and

the compounds of formula:

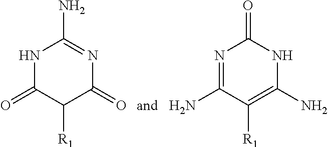

in which R₁ is a hydrogen atom or a saturated or unsaturated, optionally aromatic, linear, branched or cyclic monovalent C₁-C₆₀₀₀ hydrocarbon-based group that may comprise at least one heteroatom such as O, S, N, P or F;

(b) groups capable of forming at least three H-bonds which are complementary but different, and for example:

adenine complementary to guanine,

cytidine complementary to thymine,

triamino-s-triazine complementary to uracil and/or to succinimide and/or to glutarimide and/or to cyanuric acid and/or to thymine and/or to maleimide and/or to (di)aminopyrimidine and/or to barbituric acid, and

(acylamino)amino-s-triazine complementary to uracil and/or to succinimide and/or to glutarimide and/or to cyanuric acid and/or to thymine and/or to maleimide and/or to (di)aminopyrimidine and/or to barbituric acid.

According to at least one embodiment, the groups capable of forming at least three H-bonds are chosen from groups capable of establishing at least three H-bonds with themselves (self-complementary), for example at least four H-bonds with themselves. Among these groups, exemplary mention may be made of:

ureidopyrimidones;

ureidopyridines, carbamoylpyridines;

acylamino-s-triazines, and for example acyl(diamino)-s-triazines;

ureidotriazines;

phthalhydrazides; and

the compounds of formula:

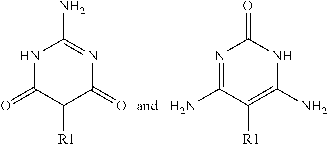

in which the R₁, R₂ and R₃ radicals have the meanings given above, for example the meanings of those narrow embodiments.

According to at least one embodiment, as examples of groups capable of forming at least three H-bonds, mention may be made of groups derived from ureidopyrimidones, and for example 2-ureidopyrimidone or 6-methyl-2-ureidopyrimidone.

The residue of the graft is constituted of a linker L comprising at least one reactive group capable of reacting with the functionalized polyalkene group(s).

The at least one reactive group may, for example, be a carboxyl group or an isocyanate group. For example, it can be an —N═C═O or —N═C═S group, and for further example it is an —N═C═O (isocyanate) group.

According to at least one embodiment, the linker L can be chosen from: phenylene; 1,4-nitrophenyl; 1,2-ethylene; 1,6-hexylene; 1,4-butylene; 1,6-(2,4,4-trimethylhexylene); 1,4-(4-methylpentylene); 1,5-(5-methylhexylene); 1,6-(6-methylheptylene); 1,5-(2,2,5-trimethylhexylene); 1,7-(3,7-dimethyloctylene); -isophorone-; 4,4′-methylenebiscyclohexylene; tolylene; 2-methyl-1,3-phenylene; 4-methyl-1,3-phenylene; and 4,4-biphenylenemethylene;

and according to at least one embodiment, the linker L can be chosen from: -isophorone-; —(CH₂)₂—; —(CH₂)₆—; —CH₂CH(CH₃)—CH₂—C(CH₃)₂—CH₂—CH₂; methylenebiscyclohexylene; and 2-methyl-1,3-phenylene.

According to at least one embodiment, the grafts are of formula (B):

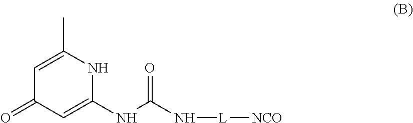

L having the same meaning as above.

According to at least one embodiment, the polyalkene-based supramolecular polymer of the disclosure is of formula (C):

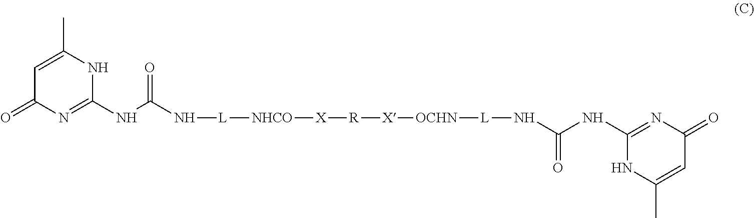

R, X, X′ and L having the meanings indicated above.

For example, in formula (C), X and X′ represent an oxygen atom.

The polyalkene-based supramolecular polymer(s) of the disclosure may also be obtained from a condensation reaction of at least one polymer (A1) comprising a polyalkene part, said polymer being functionalized with at least one reactive group (B1), with at least one molecule (A3) comprising at least one reactive group (B2), wherein the at least one molecule (A3) is such that, after reaction of the (B1) and (B2) groups, an entity capable of forming at least three H-bonds, such as at least four H-bonds, is formed.

For example, these entities have the structures (i) to (xxiv) as defined above with R₁ representing a single bond.

The polymer (A1) may for example result from the action, on a polyalkene of formula A as defined above, of compounds (A2) comprising two reactive groups (B′2) capable of reacting with the functionalized groups of the polyalkene.

These reactive groups may, for example, be carboxyl groups or isocyanate groups. For example, they can be —N═C═O or —N═C═S groups, and such as —N═C═O(isocyanate) groups.

According to at least one embodiment, the B2 groups are identical to the B′2 groups.

According to at least one embodiment, the compounds (A2) are of structure (C′) below:

B′2-L′-B′2 (C′)

the linker L′ having the same meanings as L defined above.

According to at least one embodiment, the polymers A1 are of formula (C1):

CON-L-NCO—X—R—X′—CON-L-NCO (C1)

in which L, X, X′ and R have the same meanings as above.

According to at least one embodiment, the molecule (A3) is 6-methylisocytosine of formula:

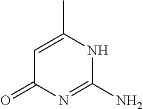

In practice, the supramolecular polymer(s) according to the disclosure may be prepared via the processes normally used by those skilled in the art for forming a urethane bond, between the free OH functions of a polyhydroxylated polyalkene and the isocyanate functions carried by the joining group. By way of illustration, a general preparation process consists in:

making sure that the polymer to be functionalized is sufficiently deprived of water;

heating the polymer comprising at least two reactive functions, such as OH, to a temperature that may range from 60° C. to 140° C. The hydroxyl number of the polymer may act as a reference in order to measure the state of progression of the reaction;

adding, directly, the graft carrying the reactive functions, for example isocyanate;

stirring the mixture, under a controlled atmosphere, at a temperature ranging from 90 to 130° C., for 1 to 24 hours;

monitoring, by infrared spectroscopy, the disappearance of the band characteristic of the isocyanates (between 2500 and 2800 cm⁻¹), so as to halt the reaction at the complete disappearance of the peak, and then allowing the final product to return to ambient temperature;

the reaction may also be monitored by quantitative determinations of the hydroxyl functions;

it is also possible to optionally add ethanol in order to make sure that the residual isocyanate functions have completely disappeared;

the mixture may be filtered if necessary.

The reaction can be carried out in the presence of a solvent, such as methyltetrahydrofuran, tetrahydrofuran, toluene or butyl acetate, or else propylene carbonate.

It is also possible to add a catalyst conventional for the formation of the urethane bond. By way of example, mention may be made of dibutyltin dilaurate.

At the end, the compound may be washed and dried, or even purified, according to the general knowledge of those skilled in the art.

According to at least one embodiment, the reaction may comprise the following stages:

(i) functionalization of the predried polyhydroxylated polyalkene polymer P with a diisocyanate according to the following reaction scheme:

OH—P—OH (1 eq)+NCO—X—NCO (1 eq)→OCN—X—NH—(O)CO—P—OC(O)—NH—X—NCO

The diisocyanate may optionally be in excess relative to the polymer P. This first stage can be carried out in the presence of a solvent, at a temperature ranging from 20° C. to 100° C.

This first stage can be followed by a period of stirring, under a controlled atmosphere, for a period ranging from 1 hour to 24 hours. The mixture may optionally be heated.

The state of progression of this first stage can be monitored by quantitative determination of the hydroxyl functions;

then

(ii) reaction of the prepolymer obtained in stage (i) with 6-methylisocytosine:

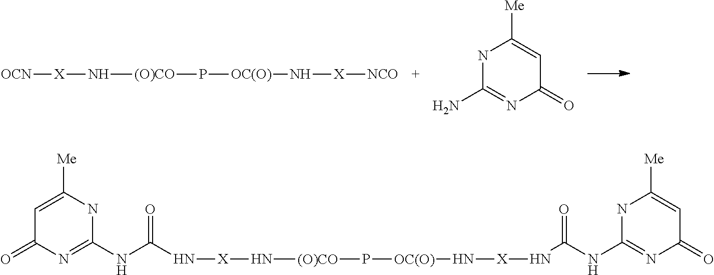

This second stage may be optionally carried out in the presence of a cosolvent such as toluene, butyl acetate or else propylene carbonate. The reaction mixture can be heated at a temperature ranging from 80° C. to 140° C. for a period of time ranging from 1 hour to 24 hours.

The presence of a catalyst may promote the production of the desired final product. Mention may be made, for example, of the use of dibutyltin dilaurate.

The reaction can be monitored by infrared spectroscopy, by monitoring the disappearance of the peak characteristic of the isocyanate between 2200 and 2300 cm⁻¹.

At the end of the reaction, ethanol can be added to the reaction medium in order to neutralize the residual isocyanate functions. The reaction mixture can optionally be filtered. For application needs, the polymer may be directly stripped in a cosmetic solvent.

The polyalkene-based supramolecular polymer(s) may be present in the composition at an amount ranging from 0.1% to 40% by weight, such as from 0.1% to 30% by weight, further such as from 0.5% to 20% by weight, and even further such as from 1% to 20% by weight, relative to the total weight of the composition.

### Block Ethylenic Copolymer

The composition according to the disclosure comprises at least one block ethylenic copolymer (also called block ethylenic polymer) comprising
at least one first block having a glass transition temperature (Tg) of greater than or equal to 40° C. which comprises at least one first monomer whose corresponding homopolymer has a glass transition temperature of greater than or equal to 40° C., and
at least one second block having a glass transition temperature of less than or equal to 20° C. which comprises at least one second monomer, whose corresponding homopolymer has a glass transition temperature of less than or equal to 20° C., wherein the at least one first block and the at least one second block are linked to one another via a random intermediate segment comprising at least one constituent monomer of the at least one first block and at least one constituent monomer of the at least one second block,
and wherein the at least one block copolymer has a polydispersity index I of greater than 2.

The term “at least” one block is intended to mean one or more blocks.

The term “block” polymer is intended to mean a polymer comprising at least 2 distinct blocks, such as at least 3 distinct blocks.

The term “ethylenic” polymer is intended to mean a polymer obtained by polymerization of monomers comprising an ethylenic unsaturation.

The block ethylenic polymer used according to the disclosure is prepared from monofunctional monomers.

This means that the block ethylenic polymer used according to the present disclosure may not comprise multifunctional monomers, which make it possible to break the linearity of a polymer in order to obtain a branched or even crosslinked polymer, according to the degree of multifunctional monomer. The block ethylenic polymer used according to the disclosure also may not comprise macromonomers (the term “macromonomer” is intended to mean a monofunctional monomer having a pendent group of polymeric nature, and for example having a molecular mass of greater than 500 g/mol, or else a polymer comprising on just one of its ends, a polymerizable end group (or comprising an ethylenic unsaturation)), which are used in the preparation of a grafted polymer.

It is pointed out that, in the above and in what follows, the terms “first” and “second” blocks do not in any way condition the order of said blocks in the structure of the block ethylenic polymer.

The first block and the second block of the block ethylenic polymer used in the disclosure may, for example, be mutually incompatible.

The term “mutually incompatible blocks” is intended to mean that the blend formed by a polymer corresponding to the first block and by a polymer corresponding to the second block is not miscible in the block polymer polymerization solvent, that is the majority amount by weight, at ambient temperature (25° C.) and atmospheric pressure (10⁵ Pa), for a content of the blend of said polymers of greater than or equal to 5% by weight, relative to the total weight of the blend of said polymers and of said polymerization solvent, it being understood that:

i) said polymers are present in the blend in a content such that the respective weight ratio ranges from 10/90 to 90/10, and that

ii) each of the polymers corresponding to the first and second blocks has an average (weight-average or number-average) molecular mass equal to that of the block ethylenic polymer ±15%.

In the case of a mixture of polymerization solvents, and should two or more solvents be present in identical mass proportions, said polymer blend is immiscible in at least one of them.

Of course, in the case of a polymerization carried out in a single solvent, the latter is the predominant solvent.

The intermediate segment (also called intermediate block) has a glass transition temperature Tg between the glass transition temperatures of the first and second blocks.

The intermediate segment, which is a block comprising at least one monomer that is a constituent of the first block and at least one monomer that is a constituent of the second block of the polymer, makes it possible to “compatibilize” these blocks.

According to at least one embodiment, the intermediate segment comprising at least one monomer that is a constituent of the first block and at least one monomer that is a constituent of the second block of the polymer is a random polymer.

According to at least one embodiment, the intermediate block is essentially derived from monomers that are constituents of the first block and of the second block.

The term “essentially” is intended to mean at least to the degree of 85%, such as at least to the degree of 90%, further such as to the degree of 95% and even further such as to the degree of 100%.

The block ethylenic polymer according to the disclosure is for example a film-forming block ethylenic polymer.

The term “film-forming” polymer is intended to mean a polymer capable of forming, by itself or in the presence of an auxiliary film-forming agent, a continuous deposit on a support, for example on keratin materials.

According to at least one embodiment, the block ethylenic polymer according to the disclosure does not comprise silicon atoms in its backbone. The term “backbone” is intended to mean the main chain of the polymer, as opposed to the pendent side chains.

According to at least one embodiment, the block ethylenic polymer according to the disclosure is water-insoluble, e.g. the block ethylenic polymer is not soluble in water or in a mixture of water and of linear or branched lower monoalcohols comprising from 2 to 5 carbon atoms, for instance ethanol, isopropanol or n-propanol, without pH modification, at an active material content of at least 1% by weight, at ambient temperature (25° C.).

According to at least one embodiment, the block ethylenic polymer according to the disclosure is not an elastomer.

The term “non-elastomeric polymer” is intended to mean a polymer which, when it is subjected to a stress intended to stretch it (for example by 30% relative to its initial length), does not return to a length substantially identical to its initial length when the stress ceases.

According to at least one embodiment, the term “non-elastomeric polymer” represents a polymer with an instantaneous recovery Ri<50% and a delayed recovery R2h<70% after having been subjected to a 30% elongation. For example, Ri is <30%, and R2h<50%.

According to at least one embodiment, the non-elastomeric nature of the polymer is determined according to the following protocol:

A polymer film is prepared by pouring a solution of the polymer into a Teflon-coated mould and then drying for 7 days in an environment conditioned at 23±5° C. and 50±10% relative humidity.

A film approximately 100 μm thick is then obtained, which is cut into rectangular specimens (for example using a punch), 15 mm wide and 80 mm long.

The specimens are subjected to a tensile stress using a machine sold under the reference Zwick, under the same temperature and humidity conditions as for the drying.

The specimens are pulled at a speed of 50 mm/min and the distance between the jaws is 50 mm, which corresponds to the initial length (I0) of the specimen.

The instantaneous recovery Ri is determined in the following manner:

the specimen is pulled by 30% (εmax), e.g. approximately 0.3 times its initial length (I0),

the stress is released by applying a return speed equal to the tensile speed, e.g. 50 mm/min, and the residual elongation of the specimen is measured as a percentage, after returning to a zero load stress (εi).

The percentage instantaneous recovery (Ri) is given by the following formula:

Ri=((εmax−εi)/εmax)×100

To determine the delayed recovery, the percentage degree of residual elongation of the specimen (ε2h) is measured 2 hours after a return to the zero load stress.

The percentage delayed recovery (R2h) is given by the following formula:

R2h=((εmax−ε2h)/εmax)×100

According to at least one embodiment, the block ethylenic polymer has an instantaneous recovery Ri of 10% and a delayed recovery R2h of 30%.

The polydispersity index of the block ethylenic polymer of the disclosure is greater than 2.

For example, the block polymer used in the compositions according to the disclosure has a polydispersity index I of greater than 2, for example ranging from 2 to 9, such as greater than or equal to 2.5, for example ranging from 2.5 to 8, and further such as greater than or equal to 2.8, and for example ranging from 2.8 to 6.

The polydispersity index I of the block ethylenic polymer is equal to the ratio of the weight-average mass Mw to the number-average mass Mn.

The weight-average molar mass (Mw) and the number-average molar mass (Mn) are determined by gel permeation liquid chromatography (THF solvent, calibration curve established with linear polystyrene standards, refractometric detector).

The weight-average mass (Mw) of the block ethylenic polymer according to the disclosure is for example less than or equal to 300,000; it ranges, for example, from 35,000 to 200,000, and for further example from 45,000 to 150,000 g/mol.

The number-average mass (Mn) of the block ethylenic polymer according to the disclosure is for example less than or equal to 70,000; it ranges, for example, from 10,000 to 60,000, and for further example from 12,000 to 50,000 g/mol.

First Block Having a Tg of Greater than or Equal to 40° C.

The first block having a Tg of greater than or equal to 40° C. has, for example, a Tg ranging from 40 to 150° C., such as greater than or equal to 50° C., ranging, for example, from 50° C. to 120° C., and further such as greater than or equal to 60° C., ranging, for example, from 60° C. to 120° C.

The glass transition temperatures indicated for the first and second blocks may be theoretical Tg values determined from the theoretical Tg values of the homopolymers of the monomers that are constituents of each of the blocks, which may be found in a reference manual such as the Polymer Handbook, 3rd edition, 1989, John Wiley, according to the following relationship, known as Fox's law:

$$1 / Tg = {\sum }_{i} ( ϖ i / Tgi ) ,$$

ω̅i being the mass fraction of the monomer i in the block under consideration and Tgi being the glass transition temperature of the homopolymer of the monomer i.

Unless otherwise indicated, the Tg values indicated for the first and second blocks in the present application are theoretical Tg values.

The difference between the glass transition temperatures of the first and second blocks is generally greater than 10° C., such as greater than 20° C., and further such as greater than 30° C.

The first block having a Tg of greater than or equal to 40° C. may be a homopolymer or a copolymer.

The first block having a Tg of greater than or equal to 40° C. may comprise at least one first monomer, whose corresponding homopolymer has a glass transition temperature of greater than or equal to 40° C. This block can also be referred to as “rigid block”.

In the case where this block is a homopolymer, it is derived from monomers whose corresponding homopolymers have glass transition temperatures of greater than or equal to 40° C. This first block can be a homopolymer constituted of a single type of monomer (the Tg of the corresponding homopolymer of which is greater than or equal to 40° C.).

In the case where the first block is a copolymer, it may comprise at least one monomer, the nature and the concentration of which is chosen such that the Tg of the resulting copolymer is greater than or equal to 40° C. The copolymer can, for example, comprise:

monomers whose corresponding homopolymers have Tg values of greater than or equal to 40° C., for example a Tg ranging from 40° C. to 150° C., such as greater than or equal to 50° C., for example ranging from 50° C. to 120° C., and further such as greater than or equal to 60° C., for example ranging from 60° C. to 120° C., and

monomers whose corresponding homopolymers have Tg values of less than 40° C., chosen from monomers having a Tg of between 20° C. and 40° C. and/or monomers having a Tg of less than or equal to 20° C., for example a Tg ranging from −100° C. to 20° C., such as less than 15° C., for example ranging from −80° C. to 15° C., and further such as less than 10° C., for example ranging from −50° C. to 0° C., as described below.

The at least one first monomer, whose corresponding homopolymer has a glass transition temperature of greater than or equal to 40° C., is for example chosen from the following monomers, also known as main monomers:

methacrylates of formula CH₂═C(CH₃)—COOR1

in which R1 represents a linear or branched unsubstituted alkyl group comprising from 1 to 4 carbon atoms, such as a methyl, ethyl, propyl or isobutyl group, or R1 represents a C₄ to C₁₂ cycloalkyl group, for example a C₈ to C₁₂ cycloalkyl group, such as isobornyl methacrylate,

acrylates of formula CH₂═CH—COOR2

in which R2 represents a C₄ to C₁₂ cycloalkyl group, such as an isobornyl group or a tert-butyl group, and

(meth)acrylamides of formula:

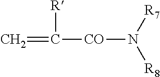

wherein R7 and R8, which may be identical or different, each represent a hydrogen atom or a linear or branched C₁ to C₁₂ alkyl group, such as an n-butyl, t-butyl, isopropyl, isohexyl, isooctyl or isononyl group; or R7 represents H and R8 represents a 1,1-dimethyl-3-oxobutyl group,

and R′ represents H or methyl. As examples of monomers, mention may be made of N-butylacrylamide, N-t-butylacrylamide, N-isopropylacrylamide, N,N-dimethyl-acrylamide and N,N-dibutylacrylamide.

The first block for example may comprise at least one acrylate monomer of formula CH₂═CH—COOR2 and at least one methacrylate monomer of formula CH₂═C(CH₃)—COOR2 in which R2 represents a C₄ to C₁₂ cycloalkyl group, for example a C₈ to C₁₂ cycloalkyl group, such as isobornyl. The monomers and the proportions thereof are for example chosen such that the glass transition temperature of the first block is greater than or equal to 40° C.

According to at least one embodiment, the first block is obtained from:

i) at least one acrylate monomer of formula CH₂═CH—COOR2 in which R2 represents a C₄ to C₁₂ cycloalkyl group, for example a C₈ to C₁₂ cycloalkyl group, such as isobornyl, and

ii) at least one methacrylate monomer of formula CH₂═C(CH₃)—COOR′2 in which R′2 represents a C₄ to C₁₂ cycloalkyl group, for example a C₈ to C₁₂ cycloalkyl group, such as isobornyl.

According to at least one embodiment, the first block is obtained from at least one acrylate monomer of formula CH₂═CH—COOR2 in which R2 represents a C₈ to C₁₂ cycloalkyl group, such as isobornyl, and from at least one methacrylate monomer of formula CH₂═C(CH₃)—COOR′2 in which R′2 represents a C₈ to C₁₂ cycloalkyl group, such as isobornyl.

For example, R2 and R′2 represent, independently or simultaneously, an isobornyl group.

According to at least one embodiment, the block ethylenic copolymer comprises from 50% to 80% by weight of isobornyl methacrylate/isobornyl acrylate, from 10% to 30% by weight of isobutyl acrylate and from 2% to 10% by weight of acrylic acid, relative to the weight of the block ethylenic polymer.

The first block may be obtained exclusively from said acrylate monomer and from said methacrylate monomer.

The acrylate monomer and the methacrylate monomer are for example in proportions by mass ranging from 30:70 to 70:30, such as from 40:60 to 60:40, further such as 50:50.

The proportion of the first block for example ranges from 20% to 90% by weight of the polymer, such as from 30% to 80%, and further such as from 60% to 80%.

According to at least one embodiment, the first block is obtained by polymerization of isobornyl methacrylate and isobornyl acrylate.

Second Block Having a Glass Transition Temperature of Less than 20° C.

The second block for example has a glass transition temperature Tg of less than or equal to 20° C., for example a Tg ranging from −100° C. to 20° C., such as less than or equal to 15° C., for example ranging from −80° C. to 15° C., and further such as less than or equal to 10° C., for example ranging from −100° C. to 10° C., for further example ranging from −30° C. to 10° C.

The second block comprises at least one second monomer, whose corresponding homopolymer has a glass transition temperature of less than or equal to 20° C.

This block can also be referred to as “flexible block”.

The at least one second monomer whose corresponding homopolymer has a Tg of less than or equal to 20° C. can be for example chosen from the following monomers:

acrylates of formula CH₂═CHCOOR3,

R3 represents an unsubstituted linear or branched C₁ to C₁₂ alkyl group, with the exception of the tert-butyl group, in which is optionally inserted at least one heteroatom chosen from 0, N and S,

methacrylates of formula CH₂═C(CH₃)—COOR4,

R4 represents an unsubstituted linear or branched C₆ to C₁₂ alkyl group in which is optionally inserted at least one heteroatom chosen from O, N and S;

vinyl esters of formula R5-CO—O—CH═CH₂,

where R5 represents a linear or branched C₄ to C₁₂ alkyl group;

C₄ to C₁₂ alkyl vinyl ethers, and

N—(C₄ to C₁₂ alkyl)acrylamides, such as N-octylacrylamide

According to at least one embodiment, the at least one monomer whose corresponding homopolymer has a Tg of less than or equal to 20° C. is chosen from isobutyl acrylate, and 2-ethylhexyl acrylate.

Each of the first and second blocks may comprise a minor proportion of at least one monomer that is a constituent of the other block.

Thus the first block may comprise at least one monomer that is a constituent of the second block and vice versa.

Each of the first and/or second blocks can comprise, in addition to the monomers indicated above, at least one other monomer, known as additional monomer, other than the main monomers mentioned above.

The nature and the amount of the at least one additional monomer is chosen such that the block in which it occurs has the desired glass transition temperature.

The at least one additional monomer is, for example, chosen from:

monomers having ethylenic unsaturation(s) comprising at least one tertiary amine function, such as 2-vinylpyridine, 4-vinylpyridine, dimethylaminoethyl methacrylate, diethylaminoethyl methacrylate, dimethylaminopropylmethacrylamide and the salts thereof,

methacrylates of formula CH₂═C(CH₃)—COOR6

in which R6 represents a linear or branched alkyl group comprising from 1 to 4 carbon atoms, such as a methyl, ethyl, propyl or isobutyl group, said alkyl group being substituted with at least one substituent chosen from hydroxyl groups (such as 2-hydroxypropyl methacrylate or 2-hydroxyethyl methacrylate) and halogen atoms (Cl, Br, I, F), such as trifluoroethyl methacrylate,

methacrylates of formula CH₂═C(CH₃)—COOR9,

R9 represents a linear or branched C₆ to C₁₂ alkyl group in which is optionally inserted at least one heteroatom chosen from O, N and S, said alkyl group being substituted with at least one substituent chosen from hydroxyl groups and halogen atoms (Cl, Br, I, F),

acrylates of formula CH₂═CHCOOR10,

R10 represents a linear or branched C₁ to C₁₂ alkyl group substituted with at least one substituent chosen from hydroxyl groups and halogen atoms (Cl, Br, I and F), such as 2-hydroxypropyl acrylate and 2-hydroxyethyl acrylate, or R10 represents a (C₁ to C₁₂ alkyl)-O—POE (polyoxyethylene) with repetition of the oxyethylene unit from 5 to 10 times, for example methoxy-POE, or R10 represents a polyoxyethylene group comprising from 5 to 10 ethylene oxide units.

For example, the first block can comprise, by way of additional monomer:

(meth)acrylic acid, such as acrylic acid,

tert-butyl acrylate,

methacrylates of formula CH₂═C(CH₃)—COOR1

in which R1 represents a linear or branched unsubstituted alkyl group comprising from 1 to 4 carbon atoms, such as a methyl, ethyl, propyl or isobutyl group,

(meth)acrylamides of formula:

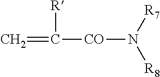

wherein R7 and R8, which may be identical or different, each represent a hydrogen atom or a linear or branched C₁ to C₁₂ alkyl group, such as an n-butyl, t-butyl, isopropyl, isohexyl, isooctyl or isononyl group; or R7 represents H and R8 represents a 1,1-dimethyl-3-oxobutyl group,

and R′ represents H or methyl. As examples of monomers, mention may be made of N-butylacrylamide, N-t-butylacrylamide, N-isopropylacrylamide, N,N-dimethylacrylamide and N,N-dibutylacrylamide,

and mixtures thereof.

The additional monomer may be present in an amount ranging from 0.5% to 30% by weight of the block ethylenic polymer. According to at least one embodiment, the block ethylenic polymer does not comprise an additional monomer.

According to at least one embodiment, the block ethylenic polymer of the disclosure comprises isobornyl acrylate and isobornyl methacrylate monomers in the first block and isobutyl acrylate and acrylic acid monomers in the second block.

For example, the block ethylenic polymer may comprise isobornyl acrylate and isobornyl methacrylate monomers in an equivalent proportion by weight in the first block and isobutyl acrylate and acrylic acid monomers in the second block.

For further example, the block ethylenic polymer may comprise isobornyl acrylate and isobornyl methacrylate monomers in an equivalent proportion by weight in the first block, and isobutyl acrylate and acrylic acid monomers in the second block, the first block representing 70% by weight of the block ethylenic polymer.

According to at least one embodiment, the block with a Tg of greater than 40° C. represents 70% by weight of the block ethylenic polymer, and the acrylic acid represents 5% by weight of the block ethylenic polymer.

According to at least one embodiment, the first block does not comprise an additional monomer.

According to at least one embodiment, the second block comprises acrylic acid by way of additional monomer. For example, the second block is may be obtained from an acrylic acid monomer and from at least one other monomer whose corresponding homopolymer has a Tg of less than or equal to 20° C.

The block ethylenic copolymer can for example comprise more than 2% by weight of acrylic acid monomer, and such as from 2% to 15% by weight, for example from 3% to 15% by weight, such as from 4% to 15% by weight, or further such as from 4% to 10% by weight of acrylic acid monomer, relative to the total weight of said copolymer.

The monomers that are constituents of the second block, and the proportions thereof, are chosen such that the glass transition temperature of the second block is less than or equal to 20° C.

### Intermediate Segment

The intermediate segment (also referred to as intermediate block) links the first block and the second block of the block ethylenic polymer used according to the present disclosure. The intermediate segment may result from the polymerization:

i) of the at least one first monomer, and optionally of the at least one additional monomer, remaining available after their polymerization to a degree of conversion of at most 90%, in order to form the first block, and

ii) of the at least one second monomer, and optionally of the at least one additional monomer, added to the reaction mixture.

The formation of the second block may be initiated when the at least one first monomer no longer reacts or is no longer incorporated in the polymer chain, either because it is all consumed or because its reactivity no longer allows it to be consumed.

Thus, the intermediate segment may comprise the available at least one first monomer, resulting from a degree of conversion of the at least one first monomer of less than or equal to 90%, during the introduction of the at least one second monomer during the synthesis of the polymer.

The intermediate segment of the block polymer can be a random polymer (may also be referred to as a random sequence), e.g. it may comprise a random distribution of the at least one first monomer and of the at least one second monomer and also optionally of the at least one additional monomer.

Thus, the intermediate segment can be a random block, just like the first block and the second block, if they are not homopolymers (e.g. if they are both formed from at least two different monomers).

### Process for Preparing the Copolymer:

The block ethylenic copolymer according to the disclosure can be prepared by free-radical polymerization according to the well-known techniques of this type of polymerization.

The free-radical polymerization can be carried out in the presence of an initiator, the nature of which is adjusted, in a known manner, according to the polymerization temperature desired and the polymerization solvent. For example, the initiator can be chosen from initiators comprising a peroxide function, oxidation/reduction couples or other radical polymerization initiators known to those skilled in the art.

For example, by way of initiator comprising a peroxide function, mention may be made of:

a. peroxyesters, such as tert-butyl peroxyacetate, tert-butyl perbenzoate, tert-butyl peroxy(2-ethylhexanoate) (TRIGONOX 21S from Akzo Nobel), or 2,5-bis(2-ethylhexanoylperoxy)-2,5-dimethylhexane (TRIGONOX® 141 from Akzo Nobel);

b. peroxydicarbonates, such as diisopropyl peroxydicarbonate;

c. peroxyketones, such as methyl ethyl ketone peroxide;

d. hydroperoxides, such as aqueous hydrogen peroxide (H₂O₂) or tert-butyl hydroperoxide;

e. diacyl peroxides, such as acetyl peroxide or benzoyl peroxide;

f. dialkyl peroxides, such as di(tert-butyl) peroxide;

g. inorganic peroxides, such as potassium peroxodisulphate (K₂S₂O₈).

By way of initiator in the form of an oxidation/reduction couple, mention may be made of the potassium thiosulphate+potassium peroxodisulphate couple, for example.

According to at least one embodiment, the initiator is chosen from organic peroxides comprising from 8 to 30 carbon atoms. For example, the initiator used is 2,5-bis(2-ethylhexanoylperoxy)-2,5-dimethylhexane, sold under the reference TRIGONOX® 141 by the company Akzo Nobel.

According to at least one embodiment, the block ethylenic copolymer used according to the disclosure is prepared by free-radical polymerization and not by controlled or living polymerization. For example, the polymerization of the block ethylenic copolymer is carried out in the absence of control agents, and for example in the absence of control agents conventionally used in living or controlled polymerization processes, such as nitroxides, alkoxyamines, dithioesters, dithiocarbamates, dithiocarbonates or xanthates, trithiocarbonates or copper-based catalysts.

As indicated above, the intermediate segment can be a random block, just like the first block and the second block if they are not homopolymers (e.g. if they are both formed from at least two different monomers).

The block ethylenic copolymer can be prepared by free-radical polymerization, and for example via a process which comprises mixing, in the same reactor, a polymerization solvent, an initiator, at least one first monomer with a glass transition of greater than or equal to 40° C. and at least one second monomer with a glass transition of less than or equal to 20° C., according to the following sequence:

a portion of the polymerization solvent and, optionally, a portion of the initiator and monomers of the first fluid addition are run into the reactor, which mixture is heated to a reaction temperature ranging from 60 to 120° C.,

said at least one first monomer with a Tg of greater than or equal to 40° C. and, optionally, a portion of the initiator are subsequently run in, in a first fluid addition, and are left to react for a time T corresponding to a degree of conversion of said monomers of at most 90%,

again polymerization initiator and said at least one second monomer with a glass transition of less than or equal to 20° C. are subsequently run into the reactor, in a second fluid addition, and are left to react for a time T′, at the end of which the degree of conversion of said monomers reaches a plateau,

the reaction mixture is brought back to ambient temperature.

According to at least one embodiment, the copolymer can be prepared by free-radical polymerization, for example via a process which comprises mixing, in the same reactor, a polymerization solvent, an initiator, an acrylic acid monomer, at least one second monomer with a glass transition of less than or equal to 20° C., at least one acrylate monomer of formula CH₂═CH—COOR2 in which R2 represents a C₄ to C₁₂ cycloalkyl group, and at least one methacrylate monomer of formula CH₂═C(CH₃)—COOR′2 in which R′2 represents a C₄ to C₁₂ cycloalkyl group, as the at least one first monomer with a Tg of greater than or equal to 40° C., according to the following sequence of stages:

a portion of the polymerization solvent and, optionally, a portion of the initiator and monomers of the first fluid addition are run into the reactor, which mixture is heated to a reaction temperature ranging from 60 to 120° C.,

said at least one acrylate monomer of formula CH₂═CH—COOR2 and said at least one methacrylate monomer of formula CH₂═C(CH₃)—COOR′2, as the at least one first monomer with a Tg of greater than or equal to 40° C., and optionally a portion of the initiator are subsequently run in, in a first fluid addition, and are left to react for a time T corresponding to a degree of conversion of said monomers of at most 90%,

again polymerization initiator, the acrylic acid monomer and said at least one second monomer with a glass transition of less than or equal to 20° C. are subsequently run in to the reactor, in a second fluid addition, and are left to react for a time T′, at the end of which the degree of conversion of said monomers reaches a plateau,

the reaction mixture is brought back to ambient temperature.

The term “polymerization solvent” is intended to mean a solvent or a mixture of solvents. Mention may for example be made, by way of polymerization solvent that can be used, of:

ketones which are liquid at ambient temperature, such as methyl ethyl ketone, methyl isobutyl ketone, diisobutyl ketone, isophorone, cyclohexanone or acetone;

propylene glycol ethers which are liquid at ambient temperature, such as propylene glycol monomethyl ether, propylene glycol monomethyl ether acetate or dipropylene glycol mono(n-butyl)ether;

short-chain esters (having a total of 3 to 8 carbon atoms), such as ethyl acetate, methyl acetate, propyl acetate, n-butyl acetate or isopentyl acetate;

ethers which are liquid at ambient temperature, such as diethyl ether, dimethyl ether or dichlorodiethyl ether;

alkanes which are liquid at ambient temperature, such as decane, heptane, dodecane, isododecane, cyclohexane or isohexadecane;

cyclic aromatic compounds which are liquid at ambient temperature, such as toluene and xylene; aldehydes which are liquid at ambient temperature, such as benzaldehyde or acetaldehyde and

mixtures thereof.

For example, the polymerization solvent is a volatile oil with a flashpoint of less than 80° C. The flashpoint is measured for example according to Standard ISO 3679.

The polymerization solvent can be chosen for example from ethyl acetate, butyl acetate, alcohols, such as isopropanol and ethanol, aliphatic alkanes such as isododecane, and mixtures thereof. For further example, the polymerization solvent is a mixture of butyl acetate and isopropanol or isododecane.

According to at least one embodiment, the block ethylenic copolymer can be prepared by free-radical polymerization according to a preparation process which comprises mixing, in the same reactor, a polymerization solvent, an initiator, at least one second monomer with a glass transition of less than or equal to 20° C., and at least one first monomer with a Tg of greater than or equal to 40° C., according to the following sequence of stages:

a portion of the polymerization solvent and, optionally, a portion of the initiator and monomers of the first fluid addition are run into the reactor, which mixture is heated to a reaction temperature ranging from 60 to 120° C.,

said at least one second monomer with a glass transition of less than or equal to 20° C. and, optionally, a portion of the initiator are subsequently run in, in a first fluid addition, and are left to react for a time T corresponding to a degree of conversion of said monomers of at most 90%,

again polymerization initiator and said at least one first monomer with a Tg of greater than or equal to 40° C. are subsequently run into the reactor, in a second fluid addition, and are left to react for a time T′, at the end of which the degree of conversion of said monomers reaches a plateau,

the reaction mixture is brought back to ambient temperature.

According to at least one embodiment, the copolymer can be prepared by free-radical polymerization according to a preparation process which comprises mixing, in the same reactor, a polymerization solvent, an initiator, an acrylic acid monomer, at least one second monomer with a glass transition of less than or equal to 20° C., at least one first monomer with a Tg of greater than or equal to 40° C., for example as the at least one first monomer with a Tg of greater than or equal to 40° C., at least one acrylate monomer of formula CH₂═CH—COOR2 in which R2 represents a C₄ to C₁₂ cycloalkyl group, and at least one methacrylate monomer of formula CH₂═C(CH₃)—COOR′2 in which R′2 represents a C₄ to C₁₂ cycloalkyl group, according to the following sequence of stages:

a portion of the polymerization solvent and, optionally, a portion of the initiator and monomers of the first fluid addition are run into the reactor, which mixture is heated to a reaction temperature ranging from 60 to 120° C.,

the acrylic acid monomer and said at least one second monomer with a glass transition of less than or equal to 20° C. and, optionally, a portion of the initiator are subsequently run in, in a first fluid addition, and are left to react for a time T corresponding to a degree of conversion of said monomers of at most 90%,

again polymerization initiator, said at least one acrylate monomer of formula CH₂═CH—COOR2 and said at least one methacrylate monomer of formula CH₂═C(CH₃)—COOR′2, as the at least one first monomer with a Tg of greater than or equal to 40° C., are subsequently run into the reactor, in a second fluid addition, and are left to react for a time T′, at the end of which the degree of conversion of said monomers reaches a plateau,

the reaction mixture is brought back to ambient temperature.

The polymerization temperature is for example of the order of 90° C.

The reaction time after the second fluid addition is for example ranging from 3 to 6 hours.

### Distillation of the Synthesis Solvent

For the use of the block ethylenic polymer in a composition according to the disclosure, and when the block ethylenic polymer is prepared in a volatile solvent or a volatile oil having a flashpoint of less than 80° C., it may be necessary to carry out a stage of complete or partial removal of said volatile solvent or oil. The operation can be carried out for example by distillation, optionally under vacuum, and addition of non-volatile hydrocarbon-based ester oil comprising at least 16 carbon atoms and having a molar mass of less than 650 g/mol.

This technique may be known to those skilled in the art. The distillation of the synthesis solvent (such as isododecane) can be carried out with simultaneous addition or in the presence in the mixture before the distillation, of a non-volatile hydrocarbon-based ester oil comprising at least 16 carbon atoms and having a molar mass of less than 650 g/mol. This stage can be carried out under hot conditions and optionally under vacuum in order to distill the maximum amount of synthesis solvent, such as isododecane, if the latter was used as polymerization solvent, or more generally in order to distill the maximum amount of volatile oil having a flashpoint of less than 80° C. The non-volatile ester oil can also be added, in part or completely, to the polymer in the volatile solvent before the distillation.

The removal of the volatile oil with a flashpoint of less than 80° C. (such as isododecane) may make it possible to limit the content of the latter in the block ethylenic copolymer solution and thus to produce a cosmetic composition comprising less than 10% by weight of the volatile solvent such as isododecane (and for example less than 5% by weight of the volatile solvent such as isododecane, relative to the total weight of the composition.

The composition according to the disclosure for example comprises an amount ranging from less than 0.5% to 40% by weight of the block ethylenic copolymer, and for example from 1% to 40% by weight, such as from 2% to 30% by weight, or further such as from 2% to 20% by weight, of the block ethylenic polymer, relative to the total weight of the composition.

### Volatile Solvent

According to the disclosure, the composition also comprises at least one volatile solvent.

As indicated herein, the term “volatile solvent” is intended to mean a compound that is liquid at ambient temperature (20° C.) and at atmospheric pressure (760 mmHg) and which has a vapor pressure at 20° C. of greater than 0.1 mmHg, and for example ranging from 0.1 to 300 mmHg, such as from 0.5 to 200 mmHg.

This volatile solvent may be water, at least one non-silicone organic solvent, at least one silicone organic solvent, or mixtures thereof.

By way of volatile non-silicone organic solvent, mention may be made of:

C₁-C₄ volatile alkanols, such as ethanol or isopropanol;

C₅-C₇ volatile alkanes, such as n-pentane, hexane, cyclopentane, 2,3-dimethylbutane, 2,2-dimethylbutane, 2-methylpentane or 3-methylpentane;

esters of liquid C₁-C₂₀ acids and of C₁-C₈ alcohols that are volatile, such as methyl acetate, n-butyl acetate, ethyl acetate, propyl acetate, isopentyl acetate or ethyl 3-ethoxypropionate;

ketones that are liquid at ambient temperature and volatile, such as methyl ethyl ketone, methyl isobutyl ketone, diisobutyl ketone, isophorone, cyclohexanone or acetone;

volatile polyols, such as propylene glycol;

volatile ethers, such as dimethoxymethane, diethoxyethane or diethyl ether;

volatile glycol ethers, such as 2-butoxyethanol, butyl diglycol, diethylene glycol monomethyl ether, propylene glycol n-butyl ether, or propylene glycol monomethyl ether acetate;

volatile hydrocarbon-based oils, such as volatile hydrocarbon-based oils comprising from 8 to 16 carbon atoms, and mixtures thereof, and for example C₈-C₁₆ branched alkanes, for instance C₈-C₁₆ isoalkanes (also known as isoparaffins), isododecane or isodecane, and, for example, the oils sold under the trade names ISOPARS® or PERMETYLS®, or mixtures thereof. Mention may also be made of isohexyl neopentanoate or isodecyl neopentanoate.

The volatile hydrocarbon-based oil may also be a linear volatile alkane chosen from linear volatile alkanes comprising from 8 to 17 carbon atoms, and for example from 9 to 15 carbon atoms, and for further example from 11 to 13 carbon atoms, such as of plant origin.

By way of example of a linear volatile alkane suitable for the disclosure, mention may be made of n-nonadecane (C9), n-decane (C10), n-undecane (C11), n-dodecane (C12), n-tridecane (C13), n-tetradecane (C14), n-pentadecane (C15) and n-hexadecane (C16);

C₄-C₁₀ volatile perfluoroalkanes, such as dodecafluoropentane, tetradecafluorohexane or decafluoropentane;

volatile perfluorocycloalkyls, such as perfluoromethylcyclopentane, 1,3-perfluorodimethylcyclohexane and perfluorodecaline, sold respectively under the names FLUTECPC1®, FLUTEC PC3® and FLUTEC PC6® by the company F2 Chemicals, and also polyfluorodimethylcyclobutane and perfluoromorpholine;

volatile fluoroalkyl or heterofluoroalkyl compounds corresponding to the formula below:

CH₃—(CH₂)ₙ—[Z]ₜ—X—CF₃

in which t is 0 or 1; n is 0, 1, 2 or 3; X is a linear or branched, divalent perfluoroalkyl radical comprising from 2 to 5 carbon atoms, and Z represents O, S or NR, R being a hydrogen atom, a —(CH₂)ₙ—CH₃ radical or a —(CF₂)ₘ—CF₃ radical, m being 2, 3, 4 or 5.

Among the volatile fluoroalkyl or heterofluoroalkyl compounds, mention may for example be made of the methoxynonafluorobutane sold under the names MSX 4518® and HFE-7100® by the company 3M, and the ethoxynonafluorobutane sold under the name HFE-7200® by the company 3M.

For example, the at least one volatile solvent is chosen in such a way that the boiling point thereof is below 200° C.

According to at least one embodiment, the non-silicone organic solvent is chosen from ethanol, isopropanol, acetone and alkanes that are liquid at 25° C. and at atmospheric pressure (760 mmHg) such as isododecane.

By way of volatile silicone solvent, mention may be made of silicone compounds with a low viscosity, chosen from linear and cyclic silicones comprising from 2 to 7 silicon atoms, these silicones optionally comprising alkyl or alkoxy groups comprising from 1 to 10 carbon atoms, such as octamethylcyclotetrasiloxane, decamethylcyclopentasiloxane, dodecamethylcyclohexasiloxane, heptamethylhexyltrisiloxane, heptamethylethyltrisiloxane, heptamethyloctyltrisiloxane, octamethyltrisiloxane, decamethyltetrasiloxane, and mixtures thereof. According to at least one embodiment, the silicone compound is chosen from cyclopentadimethylsiloxane, dodecamethylcyclohexasiloxane, octamethyltrisiloxane and decamethyltetrasiloxane.

According to at least one embodiment, the volatile silicone solvent has a viscosity of less than 50 centistokes.

For example, the volatile silicone is chosen from decamethylcyclopentasiloxane, dodecamethylcyclohexasiloxane, octamethyltrisiloxane and decamethyltetrasiloxane.

By way of example, mention may be made of the decamethylcyclopentasiloxane sold under the name DC-245 by the company Dow Corning, the dodecamethylcyclohexasiloxane sold under the name DC-246 by the company Dow Corning, the octamethyltrisiloxane sold under the name DC-200 Fluid 1 cst by the company Dow Corning and the decamethyltetrasiloxane sold under the name DC-200 Fluid 1.5 cst by the company Dow Corning.

According to at least one embodiment, the at least one volatile solvent is chosen from water, ethanol, isopropanol, acetone, the volatile alkanes as defined previously and for example isododecane, decamethylcyclopentasiloxane, octamethyltrisiloxane and decamethyltetrasiloxane.

The at least one volatile solvent may be present in the composition at an amount ranging from 0.1% to 95% by weight, relative to the total weight of the composition, such as from 1% to 90% by weight, and further such as from 5% to 90% by weight.

### Additional Silicone Compounds

According to at least one embodiment, the composition of the disclosure may also comprise at least one polysiloxane having a viscosity of greater than 100 cst, such as greater than 300 cst. The viscosity of the at least one polysiloxane can be measured according to ASTM standard D-445. Such at least one polysiloxane may be chosen from silicone oils, gums or resins, and crosslinked silicones.

By way of the at least one polysiloxane with a viscosity of greater than 100 cst, mention may for example be made of polydimethylsiloxanes; alkyl dimethicones; polyphenylmethylsiloxanes, such as phenyl dimethicones, phenyl trimethicones and vinylmethyl methicones; and also silicones modified with aliphatic and/or aromatic groups, which are optionally fluorinated, or with functional groups such as hydroxyl, thiol and/or amine groups.

Such at least one polysiloxane may be chosen from the silicones of formula (I):

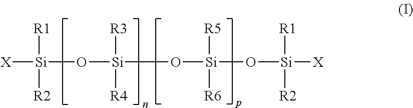

in which:

R₁, R₂, R₅ and R₆ are, which may be identical or different, an alkyl radical comprising 1 to 6 carbon atoms, R₃ and R₄ are, which may be identical or different, an alkyl radical comprising from 1 to 6 carbon atoms, a vinyl radical, an aryl radical, an aminoalkyl radical comprising from 1 to 6 carbon atoms, which is optionally substituted, a hydroxyl radical or a thioalkyl radical comprising from 1 to 6 carbon atoms, and X is an alkyl radical comprising from 1 to 6 carbon atoms, a hydroxyl radical, a vinyl radical, an aminoalkyl radical comprising from 1 to 6 carbon atoms, which is optionally substituted, or a thioalkyl radical comprising from 1 to 6 carbon atoms, n and p being integers chosen so as to obtain a viscosity of greater than 300 cst.

By way of example, mention may be made of the following polydimethylsiloxanes:

the substituents R₁ to R₆ and X represent a methyl group, such as the product sold under the name BAYSILICONE TP 3898 by the company General Electric, and the product sold under the name AK 500000 by the company Wacker,

the substituents R₁ to R₆ and X represent a methyl group, p and n are such that the molecular weight is 120 000 g/mol, such as the product sold under the name Dow Corning 200 FLUID 60000 CS by the company Dow Corning,

the substituents R₁ to R₆ and X represent a methyl group, p and n are such that the molecular weight is 250 000 g/mol, such as the product sold under the name MIRASIL DM 500,000 by the company Rhodia, and the product sold under the name Dow Corning 200 FLUID 500,000 cst by the company Dow Corning,

the substituents R₁ to R₆ represents a methyl group, the group X represents a hydroxyl group, n and p are such that the molecular weight of the polymer is 600 000 g/mol, such as the product sold under the name SGM 36 by the company Dow Corning,

dimethicones of the (polydimethylsiloxane)(methylvinylsiloxane) type, such as SE63 sold by GE Bayer Silicones, poly(dimethylsiloxane)(diphenyl)(methylvinylsiloxane) copolymers, and mixtures thereof.

When the polysiloxane comprises a fluoro group, the copolymers may be chosen from compounds of the following structure:

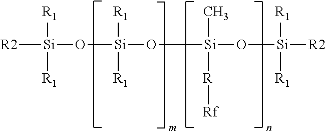

in which:

R represents a divalent, linear or branched alkyl group comprising 1 to 6 carbon atoms, such as a methyl, ethyl, propyl or butyl divalent group, Rf represents a fluoroalkyl radical, such as a perfluoroalkyl radical, comprising 1 to 12 carbon atoms, such as 1 to 9 carbon atoms, R₁ represent, independently of one another, a C₁-C₂₀ alkyl radical, a hydroxyl radical or a phenyl radical, R₂ represents R₁ or Rf,

m is chosen from 0 to 500, such as from 0 to 200, and n is chosen from 1 to 1000, such as from 1 to 500.

According to at least one embodiment, the R₁ groups are identical and represent a methyl radical.

Such polysiloxanes are for instance those sold by the company Shin Etsu under the names FL-5, FL-10, X22-821 and X22-822, or FL-100, by the company Dow Corning under the name FS-1265 FLUID, or by the company Phoenix Chemical under the PECOSIL FS range, under the names PECOSIL FSL-150, PECOSIL FSL-300, PECOSIL FSH-150, PECOSIL FSH-300, PECOSIL FSU-150 AND PECOSIL FSU-300.

The weight-average molecular mass of the polysiloxane(s) may range from 1000 to 1,500,000 g/mol, such as from 20,000 to 1,000,000 g/mol.

The polysiloxane may be a resin. The term “resin” is intended to mean a crosslinked or noncrosslinked three-dimensional structure. By way of example of a polysiloxane resin, mention may be made of silsesquioxanes and siloxysilicates.

The nomenclature of silicone resins is known as “MDTQ”, the resin being described as a function of the various siloxane monomeric units that it comprises, each of the letters “MDTQ” characterizing a type of unit.

The letter M represents the monofunctional unit of formula (CH₃)₃SiO_{1/2}, the silicon atom being linked to a single oxygen atom in the polymer comprising this unit.

The letter D signifies a difunctional (CH₃)₂SiO_{2/2} unit in which the silicon atom is linked to two oxygen atoms.

The letter T represents a trifunctional unit of formula (CH₃)SiO_{3/2}.

In the M, D and T units defined above, at least one of the methyl groups can be substituted with a group R different from the methyl group, such as a hydrocarbon-based (for example alkyl) radical comprising from 2 to 10 carbon atoms or a phenyl group or alternatively a hydroxyl group.

Finally, the letter Q signifies a tetrafunctional SiO_{4/2} unit in which the silicon atom is linked to four hydrogen atoms, themselves linked to the rest of the polymer.

Various resins having different properties can be obtained from these various units, the properties of these polymers varying according to the type of monomers (or units), to the type and number of radicals substituted, to the length of the polymer chain, to the degree of branching and to the size of the pendent chains.

By way of example of these silicone resins, mention may be made of:

siloxysilicates which can be trimethylsiloxysilicates of formula [(CH₃)₃SiO]ₓ(SiO_{4/2})_{y} (MQ units) in which x and y are integers ranging from 50 to 80,

polysilsesquioxanes of formula (CH₃SiO_{3/2})ₓ (T units) in which at least one of the methyl radicals can be substituted with a group R as defined above. For example, the number x of T units of the silsesquioxane is less than or equal to 500, such as from 50 to 500. The molecular weight of the silicone resin according to the disclosure is therefore for example from 500 to 50 000 g/mol, such as from 500 to 20 000 g/mol, and further such as from 500 to 10 000 g/mol;

polymethylsilsesquioxanes which are polysilsesquioxanes in which none of the methyl radicals are substituted with another group. Such polymethylsilsesquioxanes are described in U.S. Pat. No. 5,246,694;

polypropylsilsesquioxanes, for which the methyl radicals are replaced with propyl radicals. These compounds, and also the synthesis thereof, are for example described in WO 2005/075567;

polyphenylsilsesquioxanes, for which the methyl radicals are replaced with phenyl radicals. These compounds, and also the synthesis thereof, are for example described in US 2004/0180011.

By way of examples of commercially available polymethylsilsesquioxane resins, mention may be made of those which are marketed:

by the company Wacker under the reference RESIN MK, such as BELSIL PMS MK: polymer comprising repeating CH₃SiO_{3/2} units (T units) that may also comprise up to 1% by weight of (CH₃)₂SiO_{2/2} units (D units) and that has an average molecular weight of approximately 10 000 g/mol. It is thought that the polymer is in a “cage” and “ladder” configuration as is represented in the figures below. The average molecular weight of the units in the “cage” configuration was calculated at 536 g/mol. The majority of the polymer is in the “ladder” configuration with ethoxy groups at the ends. These ethoxy groups represent 4.5% by mass of the polymer. Since these ends can react with water, a small and variable amount of SiOH groups may also be present.

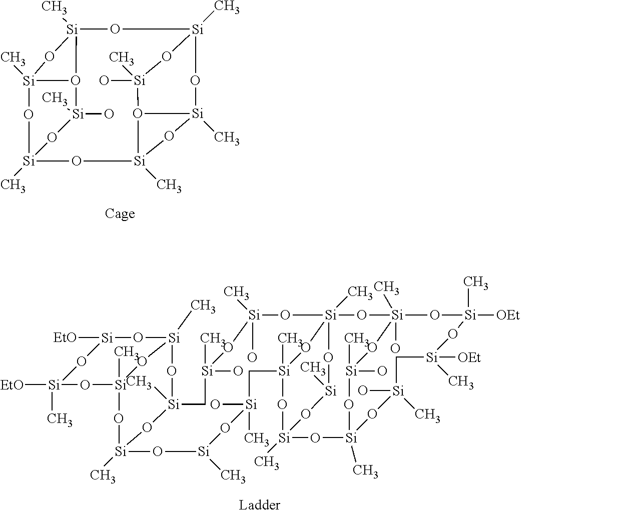

by the company Shin-Etsu under the references KR-220L, which are composed of T units of formula CH₃SiO_{3/2} and have SiOH (silanol) terminal groups, under the reference KR-242A, which comprise 98% of T units and 2% of dimethyl units D and have SiOH terminal groups, or else under the reference KR-251, comprising 88% of T units and 12% of dimethyl units D and having SiOH terminal groups.

By way of examples of commercially available polypropylsilsesquioxane resins, mention may be made of those which are marketed:

by the company Dow Corning under the reference Dow Corning 670 FIUID, which is a polypropylsilsesquioxane diluted in D5.

By way of examples of commercially available polyphenylsilsesquioxane resins, mention may be made of those which are marketed:

by the company Dow Corning under the reference Dow Corning 217 FLAKE RESIN, which is a silanol-terminated polyphenylsilsesquioxane;

by the company Wacker under the reference BELSILSPR 45 VP.

As siloxysilicate resins, mention may be made of trimethylsiloxysilicate (TMS) resins, optionally in the form of powders. Such resins are marketed under the reference SR1000 by the company General Electric or under the reference TMS 803 by the company Wacker. Mention may also be made of the trimethylsiloxysilicate resins marketed in a solvent such as cyclomethicone, sold under the name KF-7312J by the company Shin-Etsu, and DC 749 and DC 593 by the company Dow Corning.

The silicone resin according to the disclosure is for example film-forming. In fact, not all silsesquioxanes are film-forming, for example the highly polymerized polymethylsilsesquioxanes such as TOSPEARL™ from Toshiba or KMP590 from Shin-Etsu are insoluble and are not film-forming.

According to at least one embodiment, the at least one silicone resin is soluble or dispersible in the composition. For example, the silicone resins according to the disclosure are soluble in volatile silicones and organic solvents. According to at least one embodiment, the silicone resin is solid at 25° C.

The at least one silicone resin that can be used according to at least one embodiment is chosen from trimethylsiloxysilicate resins, polymethylsilsesquioxane resins and polypropylsilsesquioxane resins.

The composition of the disclosure may also contain a crosslinked silicone such as a crosslinked elastomeric organopolysiloxane, which is a high-molecular-weight silicone compound having a three-dimensional structure, with the viscoelastic properties of a flexible solid material. These organopolysiloxanes may thus be in powdered dry form, or in swollen form, in a solvent, the resulting product generally being a gel. These products may also be in a form dispersed in an aqueous solvent.

The synthesis of these organopolysiloxanes can be described in the following publications: U.S. Pat. No. 5,266,321, U.S. Pat. No. 4,742,142, U.S. Pat. No. 5,654,362, and patent application FR 2 864 784.

The elastomeric organopolysiloxanes used in the composition may be partially or totally crosslinked. They can be in the form of particles. For example, the elastomeric organopolysiloxane particles have a number-average size ranging from 0.1 to 500 μm. These particles may be of any shape, and, for example, may be spherical, flat or amorphous.

The crosslinked organopolysiloxane obtained may be a non-emulsifying compound or an emulsifying compound. The term “non-emulsifying” defines crosslinked organopolysiloxanes which do not comprise polyoxyalkylene units. The term “emulsifying” signifies crosslinked organopolysiloxane compounds comprising at least one polyoxyalkylene unit, such as polyoxyethylene or polyoxypropylene, unit.

The crosslinked organopolysiloxane particles may be conveyed in the form of a gel included in at least one hydrocarbon-based oil and/or at least one silicone oil. In these gels, the organopolysiloxane particles can be non-spherical particles. The crosslinked organopolysiloxane particles may also be in the form of a powder, such as in the form of a spherical powder.

Non-emulsifying crosslinked organopolysiloxanes are for example described in patents U.S. Pat. No. 4,970,252, U.S. Pat. No. 4,987,169, U.S. Pat. No. 5,412,004, U.S. Pat. No. 5,654,362 and U.S. Pat. No. 5,760,116, and in Japanese Application JP-A-61-194009.

As non-emulsifying crosslinked organopolysiloxanes, use may be made of those sold under the names KSG-6, KSG-15, KSG-16, KSG-18, KSG-31, KSG-32, KSG-33, KSG-41, KSG-42, KSG-43, KSG-44 and USG-103 by the company Shin-Etsu, DC 9040, DC 9041, DC 9509, DC 9505, DC 9506 and DC 9045 by the company Dow Corning, GRANSIL by the company Grant Industries, and SFE 839 by the company General Electric.

According to at least one embodiment, the emulsifying crosslinked organopolysiloxanes comprise polyoxyalkylene-modified organopolysiloxanes formed from divinyl compounds, such as polysiloxanes comprising at least two vinyl groups, which can react with Si—H bonds of a polysiloxane. Emulsifying crosslinked organopolysiloxanes are for example described in patents U.S. Pat. No. 5,236,986, U.S. Pat. No. 5,412,004, U.S. Pat. No. 5,837,793 and U.S. Pat. No. 5,811,487.

As emulsifying crosslinked organopolysiloxanes, use may be made of those marketed under the names KSG-21, KSG-20 and KSG-30 by the company Shin Etsu, and DC 9010 and DC 9011 by the company Dow Corning.

The particles of elastomeric crosslinked organopolysiloxane may also be in the form of a powder of elastomeric crosslinked organopolysiloxane coated with silicone resin, such as with silsesquioxane resin, as described, for example, in patent U.S. Pat. No. 5,538,793.

Such elastomers are sold under the names KSP-100, KSP-101, KSP-102, KSP-103, KSP-104 and KSP-105 by the company Shin Etsu.

According to at least one embodiment, the crosslinked organopolysiloxane is non-emulsifying.

The composition of the disclosure may also contain a grafted silicone polymer. As disclosed herein, the term “grafted silicone polymer” is intended to mean a polymer comprising a polysiloxane portion and a portion of a non-silicone organic chain, one of the two portions constituting the main chain of the polymer, the other being grafted onto said main chain.

The grafted silicone polymers used in the cosmetic composition according to the disclosure are for example chosen from polymers comprising a non-silicone organic backbone grafted with monomers constituting a polysiloxane, polymers comprising a polysiloxane backbone grafted with non-silicone organic monomers, and mixtures thereof.

The non-silicone organic monomers constituting the main chain of the grafted silicone polymer may be chosen from free-radical-polymerizable, ethylenically unsaturated monomers, polycondensation-polymerizable monomers, such as those forming polyamides, polyesters or polyurethanes, and ring-opening monomers such as those of the oxazoline or caprolactone type.

The polymers having a non-silicone organic backbone grafted with monomers constituting a polysiloxane, in accordance with the disclosure, can be chosen from those described in patents U.S. Pat. No. 4,693,935, U.S. Pat. No. 4,728,571 and U.S. Pat. No. 4,972,037 and patent applications EP-A-0 412 704, EP-A-0 412 707, EP-A-0 640 105 and WO 95/00578. They can be copolymers obtained by free-radical polymerization starting from ethylenically unsaturated monomers and silicone macromers having a terminal vinyl group, or else copolymers obtained by reaction of a polyolefin comprising functionalized groups and of a polysiloxane macromer having a terminal function that is reactive with said functionalized groups.

The copolymer comprising a non-silicone organic backbone grafted with monomers constituting a polysiloxane may, for example, have the following structure:

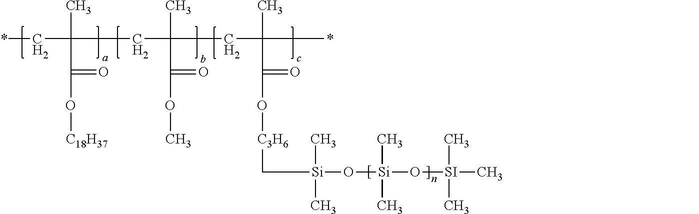

Such a polymer is marketed under the name KP 561 by Shin Etsu.

The copolymer having a non-silicone organic backbone grafted with monomers constituting a polysiloxane may also have the following structure:

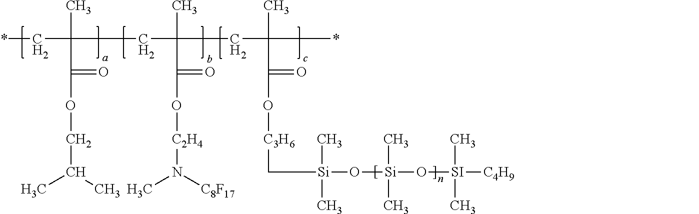

Such a polymer, Polysilicone 7, is marketed under the name SA70 by 3M.

Other copolymers having a non-silicone organic backbone grafted with monomers constituting polysiloxane may also be KP545, KP574 and KP575, marketed by Shin Etsu.

As a grafted silicone compound, mention may also be made of the isobutyl methacrylate/bis(hydroxypropyl) dimethicone acrylate copolymer sold by Grant Industries under the name GRANACRYSIL BMAS.

According to the present disclosure, the grafted silicone polymer(s), comprising a polysiloxane backbone grafted with non-silicone organic monomers, comprise(s) a main chain of silicone (or polysiloxane (≡Si—O—)ₙ) onto which is grafted, within said chain and also, optionally, at at least one of its ends, at least one organic group which does not comprise silicone.

Examples of silicone polymers corresponding to the definition are, for example, polydimethylsiloxanes (PDMSs) onto which are grafted, via a thiopropylene-type connecting chain, mixed polymer units of the poly(meth)acrylic acid type and of the poly(alkyl(meth)acrylate) type. As a compound corresponding to this definition, mention may be made of polydimethylsiloxane or polymethylsiloxane comprising methyl 3-(propylthio)acrylate/methyl methacrylate/methacrylic acid groups, or Polysilicone-8 marketed under the name VS80 by the company 3M.

Other examples of silicone polymers are for example polydimethylsiloxanes (PDMSs) onto which are grafted, via a thiopropylene-type connecting chain, polymer units of the poly(isobutyl(meth)acrylate) type.

According to at least one embodiment, the number-average molecular mass of the silicone polymers comprising a polysiloxane backbone grafted with non-silicone organic monomers, of the disclosure, ranges from 10,000 to 1,000,000, and for example from 10,000 to 100,000.

According to at least one embodiment, the grafted silicone polymers are chosen from polydimethylsiloxane-grafted alkyl methacrylate copolymer, isobutyl methacrylate/acrylic acid/silicone macromer copolymers, polydimethylsiloxane, and polymethylsiloxane comprising methyl 3-(propylthio)acrylate/methyl methacrylate/methacrylic acid groups.

The exemplary silicone compounds are silicone oils, such as those described in formula (I), and silicone resins.

When they are present in the composition of the disclosure, these silicone compounds are introduced in an amount ranging from 0.1% to 30% by weight, such as from 0.1% to 20% by weight, and further such as from 0.1% and 10% by weight, relative to the total weight of the composition.

### Pigments

According to at least one embodiment, the composition for treating the hair is a composition for dyeing keratin fibers which further comprises at least one pigment. Such a composition makes it possible to obtain persistent, coloring coatings, without degradation of the keratin fibers.

The term “pigment” is intended to mean any pigments contributing color to keratin substances. Their solubility in water at 25° C. and at atmospheric pressure (760 mmHg) can be less than 0.05% by weight, and such as less than 0.01%.

The at least one pigment is for example chosen from organic and inorganic pigments known in the art, such as those which are described in Kirk-Othmer's Encyclopaedia of Chemical Technology and in Ullmann's Encyclopaedia of Industrial Chemistry.

The at least one pigment may be in the form of a pigment powder or pigment paste. They may be coated or uncoated.

The at least one pigment may, for example, be chosen from inorganic pigments, organic pigments, lakes, special-effect pigments such as pearlescent agents, and metallic pigments or glitter.

The at least one pigment may be an inorganic pigment. The term “inorganic pigment” is intended to mean any pigment which corresponds to the definition of Ullmann's Encyclopaedia in the “Inorganic Pigment” chapter. Among the inorganic pigments that are of use in the present disclosure, mention may be made of iron or chromium oxides, manganese violet, ultramarine blue, chromium hydrate, ferric blue and titanium oxide.

The at least one pigment may be an organic pigment. The term “organic pigment” is intended to mean any pigment which corresponds to the definition of Ullmann's Encyclopaedia in the “Organic Pigment” chapter. The organic pigment may for example be chosen from nitroso, nitro, azo, xanthene, quinoline, anthraquinone, phthalocyanin, isoindolinone, isoindoline, quinacridone, perinone, perylene, diketopyrrolopyrrole, thioindigo, dioxazine, triphenylmethane and quinophthalone compounds.

For example, organic pigments may be chosen from carmine, carbon black, aniline black, azo yellow, quinacridone, phthalocyanin blue, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole and phenol derivatives, such as those described in patent FR 2 679 771.

The at least one pigment in accordance with the disclosure may also be in the form of composite pigments, as described in patent EP 1 184 426. These composite pigments may comprise for example particles comprising an inorganic core, at least one binder providing attachment of the organic pigments to the core, and at least one organic pigment which at least partially covers the core.

The organic pigment may also be a lake. The term “lake” is intended to mean dyes adsorbed onto insoluble particles, the combination thus obtained remaining insoluble during use.

The inorganic substrates onto which the dyes are adsorbed are, for example, alumina, silica, calcium sodium borosilicate, calcium aluminium borosilicate and aluminium.

Mention may be made, among the dyes, of cochineal carmine. Mention may also be made of the dyes known under the following names: D & C RED 21 (CI 45 380), D & C ORANGE 5 (CI 45 370), D & C RED 27 (CI 45 410), D & C ORANGE 10 (CI 45 425), D & C RED 3 (CI 45 430), D & C RED 4 (CI 15 510), D & C RED 33 (CI 17 200), D & C YELLOW 5 (CI 19 140), D & C YELLOW 6 (CI 15 985), D & C GREEN (CI 61 570), D & C YELLOW 10 (CI 77 002), D & C GREEN 3 (CI 42 053), D & C BLUE 1 (CI 42 090).

By way of example of lakes, mention may be made of the product known under the following name: D & C RED 7 (CI 15 850:1).

The at least one pigment can also be a special-effect pigment. The term “special-effect pigments” is intended to mean pigments which can create a colored appearance (characterized by a certain hue, a certain vividness and a certain lightness) which is not uniform and which changes as a function of the conditions of observation (light, temperature, angles of observations, etc.). They thereby contrast with colored pigments, which provide a conventional opaque, semitransparent or transparent uniform color.

There exist several types of special-effect pigments, those with a low refractive index, such as fluorescent, photochromic or thermochromic pigments, and those with a greater refractive index, such as pearlescent agents or glitter.

Mention may be made, as examples of special-effect pigments, of pearlescent pigments, such as mica coated with titanium oxide or with bismuth oxychloride, colored pearlescent pigments, such as mica coated with titanium oxide and with iron oxides, mica coated with iron oxide, mica coated with titanium oxide and, for example, with ferric blue or with chromium oxide, mica coated with titanium oxide and with an organic pigment as defined above, and also pearlescent pigments based on bismuth oxychloride. They can also be mica particles, at the surface of which at least two successive layers of metal oxides and/or of organic coloring materials are superimposed.

The pearlescent agents can for example have a yellow, pink, red, bronze, orangey, brown, gold and/or coppery color or glint.

By way of illustration of pearlescent agents that can be used in the context of the present disclosure, mention may be made of pearlescent agents of gold color sold for example by Engelhard under the name GOLD 222C (CLOISONNE), SPARKLE GOLD (TIMICA), GOLD 4504 (CHROMALITE) and MONARCH GOLD 233X (CLOISONNE); bronze pearlescent agents sold for example by Merck under the names BRONZE FINE (17384) (COLORONA) and BRONZE (17353) (COLORONA), by Eckart under the name PRESTIGE BRONZE and PRESTIGE SOFT BRONZE, and by Engelhard under the name SUPER BRONZE (CLOISONNE); orange pearlescent agents sold for example by Engelhard under the names ORANGE 363C (CLOISONNE) and ORANGE MCR 101 (Cosmica) and by Merck under the names PASSION ORANGE (Colorona) and MATTE ORANGE (17449) (Microna); brown-coloured pearlescent agents sold for example by Engelhard under the names NU ANTIQUE COPPER 340XB (Cloisonne) and BROWN CL4509 (Chromalite); pearlescent agents with a copper glint sold for example by Engelhard under the name COPPER 340A (Timica) and by Eckart under the name PRESTIGE COPPER and PRESTIGE SOFT COPPER; pearlescent agents with a red glint sold for example by Merck under the name SIENNA FINE (17386) (Colorona); pearlescent agents with a yellow glint sold for example by Engelhard under the name YELLOW (4502) (Chromalite); red-coloured pearlescent agents with a gold glint sold for example by Engelhard under the name SUNSTONE G012 (Gemtone); black pearlescent agents with a gold glint sold for example by Engelhard under the name NU ANTIQUE BRONZE 240 AB (Timica); blue pearlescent agents sold for example by Merck under the name MATTE BLUE (17433) (Microna) or DARK BLUE (117324) (Colorona); white pearlescent agents with a silvery glint sold for example by Merck under the name XIRONA SILVER; and golden green pinkish orangey pearlescent agents sold for example by Merck under the name INDIAN SUMMER (Xirona); and their mixtures.

In addition to pearlescent agents on a mica support, it is possible to envisage multilayer pigments based on synthetic substrates, such as alumina, silica, calcium sodium borosilicate, calcium aluminium borosilicate and aluminium.

Mention may also be made of pigments with an interference effect which are not attached to a substrate, such as liquid crystals (HELICONES HC from Wacker), interference holographic glitter (GEOMETRIC PIGMENTS or SPECTRA F/X from Spectratek). Special effect pigments also comprise fluorescent pigments, whether substances which are fluorescent in daylight or which produce ultraviolet fluorescence, phosphorescent pigments, photochromic pigments, thermochromic pigments and quantum dots, for example sold by Quantum Dots Corporation.

The pigment may also be in the form of a metallic pigment. The metallic pigment can be chosen from silver, aluminium, iron, chromium, nickel, molybdenum, gold, copper, zinc, tin, magnesium, steel, bronze, titanium and alloys of these metals. For example, the metallic pigment is chosen from copper, zinc, aluminium, titanium, silver, gold and alloys of these metals. Use is for example made of a metallic pigment chosen from aluminium (such as having an aluminium content of greater than or equal to 99%), copper (such as having a copper content of greater than or equal to 95%) and bronze (such as having a copper content ranging from 70% to 95% and a zinc content ranging from 5% to 30%).

The metallic pigment may also be coated with at least one “coating” layer of at least one inorganic or organic material. When the metal particle is coated in addition to or other than with the lubricant used during its production, it is for example coated with at least one layer of silicon dioxide SiO₂. These SiO₂-coated metal particles and also the preparation thereof are described, for example, in document DE 10238090.

By way of metal particles, mention may be made of aluminium particles, such as those sold under the names STARBRITE 2100 EAC® by the company Siberline and METALURE® by the company Eckart. Mention may also be made of bronze powders, such as those sold under the name PREMIER SUPER 8000 by the company Wolstenholme and under the names ROTHOFLEX, LITHOFLEX and STANDARD by the company Eckart with, for example, the SUPER PALE GOLD (D50 3-5 μm) and LITHOFLEX XA 40-03 RICH PALE GOLD (D50 3-5 μm). Mention may also be made of the metal alloy particles, for instance silica-coated bronze powders sold under the name VISIONAIRE HONEY (size 5-50 μm) and under the name VISIONAIRE AMBER (size 5-50 μm) by the company Eckart, and also those sold under the name DOROLAN 08/0 PALE GOLD (D50 7-9 μm), the SiO₂-coated aluminium powder sold under the reference VISIONAIRE SILVER SEA (size 5-50 μm) and the SiO₂-coated copper powders sold under the reference VISIONAIRE CINNAMON (size 5-50 μm) and under the reference VISIONAIRE LAVA (size 5-50 μm) by the company Eckart, and also those sold under the name DOROLAN 10/0 COPPER (D50 9-11 μm).

The variety of the pigments which can be used in the present disclosure can make it possible to obtain a rich palette of colors as well as specific optical effects, such as interference, metallic effects.

The size of the at least one pigment used in the cosmetic composition according to the present disclosure can range from 10 nm to 200 μm, such as from 20 nm to 80 μm, and further such as from 30 nm to 50 μm.

The at least one pigment can be dispersed in the composition via at least one dispersing agent.

The at least one dispersing agent serves to protect the dispersed particles from the agglomeration or flocculation thereof. The at least one dispersing agent can be chosen from a surfactant, an oligomer, and a polymer carrying at least one functionality having a strong affinity for the surface of the particles to be dispersed. For example, they can become attached physically or chemically to the surface of the pigments. These dispersants may additionally exhibit at least one functional group compatible with or soluble in the continuous medium. Use is made for example of esters of 12-hydroxystearic acid, such as, and of C₈ to C₂₀ fatty acid and of polyol, such as glycerol or diglycerol, for example the stearate of poly(12-hydroxystearic acid) with a molecular weight of approximately 750 g/mol, such as that sold under the name of SOLSPERSE 21 000 by Avecia, polyglyceryl-2 dipolyhydroxystearate (CTFA name), sold under the reference DEHYMYLS PGPH by Henkel, or polyhydroxystearic acid, such as that sold under the reference ARLACEL P100 by Uniqema, and their mixtures.

Mention may be made, as other dispersant which can be used in the compositions of the disclosure, of the quaternary ammonium derivatives of polycondensed fatty acids, such as SOLSPERSE 17 000, sold by Avecia, and polydimethylsiloxane/oxypropylene mixtures, such as those sold by Dow Corning under the references DC2-5185 and DC2-5225 C.

The at least one pigment used in the cosmetic composition according to the disclosure can be surface-treated with at least one organic agent.

Thus, the surface-pretreated pigments that are of use in the context of the disclosure can be pigments which have been completely or partially subjected to a surface treatment of chemical, electronic, electrochemical, mechanicochemical or mechanical nature, with at least one organic agent, such as those described for example in Cosmetics and Toiletries, February 1990, Vol. 105, p. 53-64, before being dispersed in the composition in accordance with the disclosure. The at least one organic agent may, for example, be chosen from amino acids; waxes, for example carnauba wax and beeswax; fatty acids, fatty alcohols and their derivatives, such as stearic acid, hydroxystearic acid, stearyl alcohol, hydroxystearyl alcohol, lauric acid and their derivatives; anionic surfactants; lecithins; sodium, potassium, magnesium, iron, titanium, zinc or aluminium salts of fatty acids, for example aluminium stearate or aluminium laurate; metal alkoxides; polysaccharides, for example chitosan, cellulose and its derivatives; polyethylene; (meth)acrylic polymers, for example poly(methyl methacrylate)s; polymers and copolymers comprising acrylate units; proteins; alkanolamines, silicone compounds, for example silicones, polydimethylsiloxanes, alkoxysilanes, alkylsilanes or siloxysilicates; fluorinated organic compounds, for example perfluoroalkyl ethers; and fluorosilicone compounds.

The surface-treated pigments that are of use in the cosmetic composition according to the disclosure may also have been treated with a mixture of these compounds and/or have undergone several surface treatments.

The surface-treated pigments that are of use in the context of the present disclosure may be prepared according to surface-treatment techniques well known to those skilled in the art or found as such commercially.

For example, the surface-treated pigments are covered with an organic layer.

The organic agent with which the pigments are treated can be deposited on the pigments by evaporation of solvent, chemical reaction between the molecules of the surface agent or creation of a covalent bond between the surface agent and the pigments.

The surface treatment may thus be carried out, for example, by chemical reaction of a surface agent with the surface of the pigments and creation of a covalent bond between the surface agent and the pigments or fillers. This method is for example described in patent U.S. Pat. No. 4,578,266.

For example, use can be made of an organic agent covalently bonded to the pigments.

The agent for the surface treatment can represent from 0.1% to 50% by weight, such as from 0.5% to 30% by weight, and further such as from 1% to 10% by weight, relative to the total weight of the surface-treated pigments.

When they are present, the amount of pigments can range from 0.1% to 40% by weight, such as from 0.5% to 20% by weight, relative to the total weight of the composition.

### Other Additives

When the polymer has a glass transition temperature that is too high for the desired use, at least one plasticizer may be combined therewith so as to reduce this temperature of the mixture used. The at least one plasticizer may be chosen from plasticizers normally used in the field of application, and for example from compounds that can be solvents for the polymer.

For example, the at least one plasticizer may have a molecular mass of less than or equal to 5000 g/mol, such as less than or equal to 2000 g/mol, further such as less than or equal to 1000 g/mol. The at least one plasticizer for example has a molecular mass of greater than or equal to 100 g/mol.

Thus, the composition may also comprise at least one plasticizer. for example, mention may be made, alone or as a mixture, of the usual plasticizers, such as:

glycols and derivatives thereof, such as diethylene glycol ethyl ether, diethylene glycol methyl ether, diethylene glycol butyl ether or else diethylene glycol hexyl ether, ethylene glycol ethyl ether, ethylene glycol butyl ether or ethylene glycol hexyl ether;

polyethylene glycols, polypropylene glycols, polyethylene glycol/polypropylene glycol copolymers and mixtures thereof, in particular high-molecular-weight polypropylene glycols having, for example, a molecular mass ranging from 500 to 15 000, such as, for example:

glycol esters;

esters of acids, for example carboxylic acids, such as citrates, phthalates, adipates, carbonates, tartrates, phosphates or sebacates;

esters derived from the reaction of a monocarboxylic acid of formula R₁₁COOH with a diol of formula HOR₁₂OH where R₁₁ and R₁₂, which may be identical or different, represent a saturated or unsaturated, linear, branched or cyclic, hydrocarbon-based chain for example comprising from 3 to 15 carbon atoms, optionally comprising at least one heteroatom such as N, O or S, such as the monoester resulting from the reaction of isobutyric acid and octanediol, such as 2,2,4-trimethylpentane-1,3-diol, for instance the product sold under the reference TEXANOL ESTER ALCOHOL by the company Eastman Chemical;

oxyethylenated derivatives, such as oxyethylenated oils, for example plant oils, such as castor oil;

and mixtures thereof.

The composition according to the disclosure may comprise at least one thickener chosen from polymeric thickeners and inorganic thickeners.

The at least one thickener may be inorganic or organic, and polymeric or non-polymeric. The at least one thickener may be chosen to thicken an aqueous phase or a fatty phase of the composition, as appropriate.

The term “thickener” is intended to mean a compound that modifies the rheology of the medium into which it is incorporated by increasing by at least 100 cps the viscosity of the medium at 25° C. and at a shear rate of 1 s⁻¹. This viscosity can be measured using a cone/plate viscometer (Haake R600 rheometer, or the like).

The aqueous-medium thickener may be chosen from:

hydrophilic clays,

hydrophilic fumed silica,

water-soluble cellulose-based thickeners, such as hydroxyethylcellulose, methylcellulose or hydroxypropylcellulose. Among these, mention may for example be made of the gums sold under the name CELLOSIZE QP 4400 H by the company Amerchol,

nonionic guar gums comprising C₁-C₆ hydroxyalkyl groups. By way of example, mention may be made of hydroxymethyl, hydroxypropyl and hydroxybutyl groups. Such guar gums are for example sold under the trade names JAGUAR® HP8, JAGUAR® HP60, JAGUAR® HP120 and JAGUAR® HP105 by the company Meyhall or under the name GALACTASOL 40H4FD2 by the company Aqualon,

carrageenans,

locust bean gum, scleroglucan gum, gellan gum, rhamsan gum or karaya gum,

alginates, maltodextrins, starch and derivatives thereof, and hyaluronic acid and salts thereof,

polyglyceryl(meth)acrylate polymers sold under the names HISPAGEL or LUBRAGEL by the companies Hispano Quimica or Guardian,

polyvinyl alcohol,

crosslinked acrylamide polymers and copolymers, such as those sold under the names PAS 5161 or BOZEPOL C by the company Hoechst, SEPIGEL 305 by the company Seppic or by the company Allied Colloid, or

the crosslinked methacryloyloxyethyltrimethylammonium chloride homopolymers sold under the name SALCARE SC95 by the company Allied Colloid,

associative polymers, and for example associative polyurethanes.

Such thickeners are for example described in application EP-A-1400234.

The oily-medium thickener may be chosen from:

organophilic clays;

hydrophobic fumed silicas;

alkyl guar gums (with a C₁-C₆ alkyl group), such as those described in EP-A-708114;

oil-gelling polymers, for instance triblock polymers or star polymers resulting from the polymerization or copolymerization of at least one monomer containing an ethylenic group, for instance the polymers sold under the name KRATON;

polymers with a weight-average molecular mass of less than 100 000, comprising a) a polymer backbone comprising hydrocarbon-based repeating units comprising at least one heteroatom, and optionally b) at least one pendent fatty chain and/or at least one terminal fatty chain, which are optionally functionalized, comprising from 6 to 120 carbon atoms and being linked to these hydrocarbon-based units, as described in PCT Application Publications WO 02/056847 and WO02/47619; for example, polyamide resins (such as those comprising alkyl groups comprising from 12 to 22 carbon atoms) such as those described in U.S. Pat. No. 5,783,657;

the silicone-based polyamide resins as described in patent application EP-A-1266647 and in the French patent application filed under No. 0 216 039.

Such thickeners are for example described in application EP-A-1400234.

The at least one thickener may be an organic gelling agent, i.e. an agent comprising at least one organic compound. The organogelling agents may be chosen from those described in PCT Application Publication WO03/105788.

For example, the polymeric thickener present in the composition according to the disclosure is an amorphous polymer formed by polymerization of an olefin. The olefin may for example be an elastomeric ethylenically unsaturated monomer.

As examples of olefins, mention may be made of ethylenic carbide monomers, such as comprising one or two ethylenic unsaturations, and comprising from 2 to 5 carbon atoms, such as ethylene, propylene, butadiene or isoprene.

The polymeric thickener is capable of thickening or gelling the composition. The term “amorphous polymer” is intended to mean a polymer that does not have a crystalline form. The polymeric thickener may also be film-forming.

The polymeric thickener may for example be a diblock, triblock, multiblock, radial or star copolymer, or mixtures thereof.

Such polymeric thickeners are for example described in US2002/005562 and U.S. Pat. No. 5,221,534.

According to at least one embodiment, the polymeric thickener is an amorphous block copolymer of styrene and of olefin.

The polymeric thickener is for example hydrogenated to reduce the residual ethylenic unsaturations after the polymerization of the monomers.

For example, the polymeric thickener is an optionally hydrogenated copolymer, comprising styrene blocks and ethylene/C₃-C₄ alkylene blocks.

As diblock copolymers, that are for example hydrogenated, mention may be made of styrene-ethylene/propylene copolymers and styrene-ethylene/butadiene copolymers. Diblock polymers are for example sold under the name KRATON® G1701 E by the company Kraton Polymers.

As triblock copolymers, that are for example hydrogenated, mention may be made of styrene-ethylene/propylene-styrene copolymers, styrene-ethylene/butadiene-styrene copolymers, styrene-isoprene-styrene copolymers and styrene-butadiene-styrene copolymers. Triblock polymers are for example sold under the names KRATON® G1650, KRATON® G1652, KRATON® D1101, KRATON® D1102 and KRATON® D1160 by the company Kraton Polymers.

Use may also be made of a mixture of styrene-butylene/ethylene-styrene triblock hydrogenated copolymer and of ethylene-propylene-styrene hydrogenated star polymer, such a mixture being for example in isododecane. Such mixtures are, for example, sold by the company Penreco under the trade names VERSAGEL® M5960 and VERSAGEL® M5670.

According to at least one embodiment, a diblock copolymer such as those described previously, for example a styrene-ethylene/propylene diblock copolymer, is used as polymeric thickener.

According to at least one embodiment, the organic clays are clays modified with chemical compounds that make the clay capable of swelling.

Clays can be products already known per se, which are described, for example, in the book “Minéralogie des argiles” [Clay Mineralogy], S. Caillère, S. Hénin, M. Rautureau, 2^{nd} edition 1982, Masson, the teaching of which is included herein by way of reference.

Clays can be silicates comprising a cation that may be chosen from calcium, magnesium, aluminium, sodium, potassium and lithium cations, and mixtures thereof.

By way of examples of such products, mention may be made of clays of the smectite family, such as montmorillonites, hectorites, bentonites, beidellites and saponites, and also of the family of vermiculites, stevensite and chlorites.

These clays may be of natural or synthetic origin. For example, clays that are cosmetically compatible and acceptable with keratin materials may be used.

The organophilic clay may be chosen from montmorillonite, bentonite, hectorite, attapulgite and sepiolite, and mixtures thereof. The clay is for example a bentonite or a hectorite.

These clays may be modified with at least one chemical compound chosen from quaternary amines, tertiary amines, amine acetates, imidazolines, amine soaps, fatty sulphates, alkyl aryl sulphonates and amine oxides.

As organophilic clays, mention may be made of quaternium-18 bentonites such as those sold under the names BENTONE 3, BENTONE 38 and BENTONE 38V by the company Rheox, TIXOGEL VP by the company United Catalyst, CLAYTONE 34, CLAYTONE 40 and CLAYTONE XL by the company Southern Clay; stearalkonium bentonites such as those sold under the names BENTONE 27 by the company Rheox, TIXOGEL LG by the company United Catalyst and CLAYTONE AF and CLAYTONE APA by the company Southern Clay; quaternium-18/benzalkonium bentonites such as those sold under the names CLAYTONE HT and CLAYTONE PS by the company Southern Clay.

The fumed silicas may be obtained by high-temperature hydrolysis of a volatile silicon compound in an oxhydric flame, producing a finely divided silica. This process makes it possible for example to obtain hydrophilic silicas comprising a large number of silanol groups at their surface. Such hydrophilic silicas are, for example, marketed under the names AEROSIL 130®, AEROSIL 200®, AEROSIL 255®, AEROSIL 300® and AEROSIL 380® by the company Degussa, and CAB-O-SIL HS-5®, CAB-O-SILEH-5®, CAB-O-SILLM-130®, CAB-O-SILMS-55® and CAB-O-SILM-5® by the company Cabot.

It is possible to chemically modify the surface of said silica, via a chemical reaction generating a reduction in the number of silanol groups. It is for example possible to substitute silanol groups with hydrophobic groups; a hydrophobic silica can then obtained.

The hydrophobic groups for example may be:

trimethylsiloxyl groups, which are obtained for example by treating fumed silica in the presence of hexamethyldisilazane. Silicas thus treated are known as “silica silylate” according to the CTFA (6^{th} edition, 1995). They are, for example, marketed under the references AEROSIL R812® by the company Degussa and CAB-O-SIL TS-530® by the company Cabot;

dimethylsilyloxyl or polydimethylsiloxane groups, which are for example obtained by treating fumed silica in the presence of polydimethylsiloxane or of dimethyl-dichlorosilane. Silicas thus treated are known as “silica dimethyl silylate” according to the CTFA (6^{th} edition, 1995). They are, for example, marketed under the references AEROSIL R972® and AEROSIL R974® by the company Degussa, and CAB-O-SIL TS-610® and CAB-O-SIL TS-720® by the company Cabot.

The fumed silica for example has a particle size that may be nanometric to micrometric, for example ranging from 5 to 200 nm.

An organomodified bentonite or hectorite is for example used as inorganic thickener.

The at least one thickener may be present in the composition in a total content ranging from 0.1% to 10% by weight, relative to the total weight of the composition, such as ranging from 0.5% to 7% by weight, and further such as ranging from 0.5% to 5% by weight, relative to the total weight of the composition.

The composition in accordance with the disclosure may also comprise at least one agent that may be conventionally used in cosmetics, chosen, for example, from reducing agents, fatty substances, softeners, antifoams, moisturizers, UV-screening agents, inorganic colloids, peptizers, fragrances, anionic, cationic, nonionic or amphoteric surfactants, proteins, vitamins, propellants, oxyethylenated or non-oxyethylenated waxes, paraffins, C₁₀-C₃₀ fatty acids such as stearic acid or lauric acid, C₁₀-C₃₀ fatty amides such as lauric diethanolamide, and anionic, cationic, nonionic and amphoteric polymers.

The above additives are for example present in an amount for each of them ranging from 0.01% to 20% by weight, relative to the total weight of the composition.

Of course, those skilled in the art will take care to select this or these optional additive(s) in such a way that the beneficial properties that may be associated with the formation of the coating in accordance with the disclosure are not, or are not substantially, impaired.

The composition according to the disclosure may for example be in the form of a suspension, a dispersion, a solution, a gel, an emulsion, such as an oil-in-water (O/W) or water-in-oil (W/O) emulsion or a multiple emulsion (W/O/W or polyol/O/W or O/W/O), in the form of a cream, a mousse, a stick, a dispersion of vesicles, such as of ionic or nonionic lipids, a two-phase or multi-phase lotion, a spray, a powder or a paste. The composition may also be in the form of a lacquer.

Those skilled in the art may select the appropriate galenical form, and also the method for the preparation thereof, on the basis of their general knowledge, taking into account for example firstly the nature of the constituents used, such as their solubility in the carrier, and secondly the intended use of the composition.

The composition may be an anhydrous composition, e.g. a composition comprising less than 2% by weight of water, or such as less than 0.5% of water, further such as free of water, the water not being added during the preparation of the composition, but corresponding to the residual water introduced by the mixed ingredients.

The composition described above may be used on dry or wet hair and also on all types of fair or dark, natural or dyed, permanent-waved, bleached or relaxed hair.

According to at least one embodiment of the process for treating keratin fibers, the hair is washed before application of the composition described above. For example, the composition is applied to clean hair.

The application may be carried out on dry or wet hair.

The application to the hair may be performed, for example, using a comb, a fine brush, a coarse brush or the fingers.

According to at least one embodiment, the application of the composition is subsequently followed by drying at a temperature above 40° C. For example, this temperature is above 45° C. For further example, this temperature is above 45° C. and below 220° C.

The drying can be carried out immediately after the application or after a leave-in time that can range from 1 minute to 30 minutes.

According to at least one embodiment, in addition to supplying heat, the hair is dried using a flow of air. This flow of air during drying may make it possible to improve the individualization of the coating.

During drying, a mechanical action on the locks may be exerted, such as combing, brushing or running the fingers through.

The drying step of the process for treating keratin fibers may be performed with a hood, a hairdryer, a smoothing iron, etc.

When the drying step is performed with a hood or a hairdryer, the drying temperature can range from 40 to 110° C., such as from 50 to 90° C.

When the drying step is performed with a smoothing iron, the drying temperature can range from 110 to 220° C., such as from 140 to 200° C.

Once the drying is complete, a final rinse or shampoo wash may optionally be performed.

The following examples serve to illustrate the disclosure without limiting the scope thereof.

### EXAMPLES

### Example 1

### Treatment Composition

The following compositions were prepared:

| | | | A′ (not | |
|---|---|---|---|---|
| | | | part of the | |
| Composition | A | | disclosure) | |
| Supramolecular polymer obtained using | 40 | g | 40 | g |
| GI2000 as functionalized polyalkene of | | | | |
| formula A and a graft of formula B in | | | | |
| which L denotes an isophorone radical, | | | | |
| at 25% in isododecane, prepared as | | | | |
| described below | | | | |
| Poly(isobornyl methacrylate-co-isobornyl | 8 | g | — | |
| acrylate-co-isobutyl acrylate-co-acrylic | | | | |
| acid) at 50% in isododecane prepared as | | | | |
| described below * Trimethylsiloxysilicate | | | | |
| Resin sold by Momentive Performance | | | | |
| Materials under the name SR1000 | | | | |
| Trimethylsiloxysilicate resin sold by | 1.5 | g | 1.5 | g |
| Momentive Performance Materials under | | | | |
| the name SR1000 | | | | |
| Ethanol | 5 | g | 5 | g |
| Isododecane | Qs 100 | g | Qs 100 | g |

0.5 g of composition A was applied to a lock of 2.5 g of clean, wet hair with a tone depth of 4. After a leave-in time of 2 minutes, the lock was dried with a hairdryer at a temperature of 80° C. for 2 minutes. A lock was obtained, the hairs of which were individualized and had body; the volumization obtained was persistent with respect to shampooing.

The lock to which composition A′ had been applied had less resistance to fatty substances, such as to sebum, than the lock to which composition A had been applied.

### Example 2

### Treatment Composition

The following composition was prepared:

| | |
|---|---|
| Composition | B |

| | | |
|---|---|---|
| Supramolecular polymer obtained using GI2000 | 40 | g |
| as functionalized polyalkene of formula A and a | | |
| graft of formula B in which L denotes an | | |
| isophorone radical, at 25% in isododecane, | | |
| prepared as described below* | | |
| Alpha, omega-dihydroxylated poly- | 14 | g |
| dimethylsiloxane/cyclopentadimethylsiloxane | | |
| mixture (14.7/85.3) sold by Dow Corning under | | |
| the name DC1501 FLUID | | |
| Poly(isobornyl methacrylate-co-isobornyl acrylate- | 8 | g |
| co-isobutyl acrylate-co-acrylic acid) at 50% in | | |
| isododecane prepared as described below* | | |
| Trimethylsiloxysilicate resin sold by Momentive | 1.5 | g |
| Performance Materials under the name SR1000 | | |
| Ethanol | 5 | g |
| Isododecane | Qs 100 | g |

*the concentration indicated corresponds to the pure polymer.

0.5 g of composition B was applied to a lock of 2.5 g of clean, wet hair with a tone depth of 4. After a leave-in time of 2 minutes, the lock was dried with a hairdryer at a temperature of 80° C. for 2 minutes. A lock was obtained, the hairs of which were individualized and had body; the volumization obtained was persistent with respect to shampooing. This lock also had good resistance to fatty substances.

### Example 3

### Dyeing Composition

The following compositions are prepared:

| | | | C′ (not | |
|---|---|---|---|---|
| | | | part of the | |
| Composition | C | | disclosure) | |
| Supramolecular polymer obtained using | 32 | g | 32 | g |
| GI2000 as functionalized polyalkene of | | | | |
| formula A and a graft of formula B in which | | | | |
| L denotes an isophorone radical, at 25% in | | | | |
| isododecane, prepared as described below* | | | | |
| Alpha, omega-dihydroxylated poly- | 14 | g | 14 | g |
| dimethylsiloxane/cyclopentadimethylsiloxane | | | | |
| mixture (14.7/85.3) sold by Dow Corning | | | | |
| under the name DC1501 FLUID | | | | |
| Brown iron oxide-coated mica | 8 | g | 8 | g |
| pearlescent agent sold by Eckart under the | | | | |
| name PRESTIGE SOFT BRONZE | | | | |
| Poly(isobornyl methacrylate-co- | 8 | g | — | |
| isobornyl acrylate-co-isobutyl acrylate-co- | | | | |
| acrylic acid) at 50% in isododecane prepared | | | | |
| as described below* | | | | |
| Ethanol | 5 | g | 5 | g |
| Isododecane | Qs 100 | g | Qs 100 | g |

0.6 g of composition C was applied to a lock of 1 g of clean, wet hair with a tone depth of 4. After a leave-in time of 2 minutes, the lock was dried with a hairdryer at a temperature of 80° C. for 2 minutes. A colored lock was obtained, the hairs of which were individualized and the color of which was very homogeneous and persistent with respect to shampooing.

The lock to which composition C′ had been applied had less resistance to shampooing and less resistance to fatty substances, such as to sebum, than the lock to which composition C had been applied.

### Example 4

The following compositions were prepared:

| Composition | D | | D′ | |
|---|---|---|---|---|
| Supramolecular polymer obtained using | 32 | g | — | |
| GI3000 as functionalized polyalkene of | | | | |
| formula A and a graft of formula B in | | | | |
| which L denotes a hexamethylene radical, | | | | |
| at 25% in isododecane, prepared as | | | | |
| described below* | | | | |
| Supramolecular polymer obtained using | — | | 32 | g |
| GI2000 as functionalized polyalkene of | | | | |
| formula A and a graft of formula B in which | | | | |
| L denotes an isophorone radical, at 25% in | | | | |
| isododecane, prepared as described below* | | | | |
| Alpha, omega-dihydroxylated poly- | 14 | g | 14 | g |
| dimethylsiloxane/cyclopentadimethylsiloxane | | | | |
| mixture (14.7/85.3) sold by Dow Corning | | | | |
| under the name DC1501 FLUID | | | | |
| Brown iron oxide-coated mica pearlescent | 8 | g | 8 | g |
| agent sold by Eckart under the name | | | | |
| PRESTIGE SOFT BRONZE | | | | |
| Poly(isobornyl methacrylate-co-isobornyl | 8 | g | 8 | g |
| acrylate-co-isobutyl acrylate-co-acrylic acid) | | | | |
| at 50% in isododecane prepared as described | | | | |
| below* | | | | |
| Polymethylsilsesquioxane sold under the | 3 | g | 3 | g |
| name WACKER BELSIL PMS MK | | | | |
| POWDER by the company Wacker | | | | |
| Trimethylsiloxysilicate resin sold by | 2 | g | 2 | g |
| Momentive Performance Materials under | | | | |
| the name SR1000 | | | | |
| Ethanol | 5 | g | 5 | g |
| Isododecane | Qs 100 | g | Qs 100 | g |

0.6 g of composition D or D′ was applied to a lock of 1 g of clean, wet hair with a tone depth of 4. After a leave-in time of 2 minutes, the lock was dried with a hairdryer at a temperature of 80° C. for 2 minutes. A colored lock was obtained, the hairs of which were individualized and the color of which was very homogeneous and persistent with respect to shampooing.

The supramolecular polymer used in Examples 1, 2, 3 and 4 (Composition D′) above was synthesized in the following way.

106.1 g of GI2000 polymer marketed by the company Nisso, in the presence of 22 mg of catalyst, dibutyltin dilaurate, were heated at 80° C. under vacuum for 2 hours. The temperature of the mixture was brought down to 20° C. under argon, followed by the addition of 10 ml of isododecane. 19.3 g of isophorone diisocyanate were added. The mixture was stirred for 16 hours at 20° C., under a controlled atmosphere, and was then heated to 120° C., followed by the addition of 25 ml of propylene carbonate. 12 g of 6-methylisocytosine were added. This resulted in a homogenous white suspension. This suspension was heated to 140° C. and was stirred at this temperature for 6 hours. The reaction was monitored by infrared spectroscopy, until total disappearance of the peak characteristic of the isocyanates (2250 cm⁻¹). The mixture was then brought back down to 30° C., and 400 ml of heptane, 200 ml of THF and 50 ml of ethanol were added to the mixture, before filtration through celite. Stripping with isododecane made it possible to obtain the polymer at a 20% solids content. The polymer was characterized by GC (Mn=7000 with a PI of 2.05).

The supramolecular polymer used in Example 4 (Composition D) above was synthesized in the following way.

100 g of GI3000 polymer marketed by the company Nisso were dried at 80° C. under vacuum overnight. This polymer was dissolved in 400 ml of anhydrous toluene. 25 μl of catalyst, dibutyltin dilaurate, were added to the reaction mixture. The medium was heated at 80° C. and mixed until a homogeneous solution was obtained. 15 g of isocyanate-functionalized molecule having the following structure:

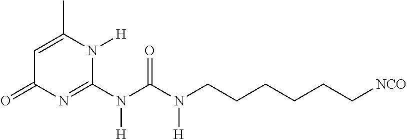

were added in solution in 300 ml of anhydrous toluene, under a controlled atmosphere at 40° C. The reaction mixture was heated to 100° C. and stirred at this temperature for 4 hours. The reaction was monitored by infrared spectroscopy, with monitoring of the total disappearance of the peak characteristic of the isocyanates at 2260 cm⁻¹. At the end of the reaction, 100 ml of ethanol were added in order to remove any trace of residual isocyanate. The mixture was filtered after having added isododecane in order to make the solution less viscous. The polymer solution was then directly stripped with isododecane. The final polymer was obtained at a 21% solids content in isododecane and was characterized by GC (Mn=6400 and a polydispersity index PI of 1.85) and ¹H NMR (spectrum in accordance with what is expected).

The poly(isobornyl acrylate/isobornyl methacrylate/isobutyl acrylate/acrylic acid) copolymer used in the examples was synthesized according to the following procedure.

300 g of isododecane were introduced into a 1-litre reactor, and then the temperature was increased so as to go from ambient temperature (25° C.) to 90° C. in 1 hour. 105 g of isobornyl methacrylate (manufactured by Arkema), 105 g of isobornyl acrylate (manufactured by Arkema) and 1.8 g of 2,5-bis(2-ethylhexanoylperoxy)-2,5-dimethylhexane (TRIGONOX® 141 from Akzo Nobel) were then added, at 90° C. and in 1 hour.

The mixture was kept at 90° C. for 1 hour 30 minutes.
75 g of isobutyl acrylate (manufactured by Fluka), 15 g of acrylic acid and 1.2 g of 2,5-bis(2-ethylhexanoylperoxy)-2,5-dimethylhexane were then introduced into the previous mixture, still at 90° C., and in 30 minutes.
The mixture was maintained at 90° C. for 3 hours, and then the whole was cooled.
A solution containing 50% of active material in terms of the block ethylenic polymer, in isododecane, was obtained.

Thus, a block ethylenic polymer comprising a poly(isobornyl acrylate/isobornyl methacrylate) rigid first block having a Tg of 110° C., a poly(isobutyl acrylate/acrylic acid) flexible second block having a Tg of −9° C. and an intermediate block which is an isobornyl acrylate/isobornyl methacrylate/isobutyl acrylate/acrylic acid random polymer, was obtained.

What is claimed is:

1. A composition for treating keratin fibers, comprising:
at least one polyalkene-based supramolecular polymer,
at least one block ethylenic copolymer comprising
at least one first block having a glass transition temperature (Tg) of greater than or equal to 40° C. which comprises at least one first monomer whose corresponding homopolymer has a glass transition temperature of greater than or equal to 40° C., and
at least one second block having a glass transition temperature of less than or equal to 20° C. which comprises at least one second monomer, whose corresponding homopolymer has a glass transition temperature of less than or equal to 20° C.,
wherein the at least one first block and the at least one second block are linked to one another via a random intermediate segment comprising at least one constituent monomer of the at least one first block and at least one constituent monomer of the at least one second block,
and wherein the at least one block copolymer has a polydispersity index I of greater than 2, and
at least one volatile solvent.

2. The composition according to claim 1, wherein the at least one polyalkene of the at least one polyalkene-based supramolecular polymer is chosen from poly(ethylenebutylene)s, hydrogenated polybutadienes, nonhydrogenated polybutadienes, hydrogenated polyisoprenes, and nonhydrogenated polyisoprenes.

3. The composition according to claim 1, wherein the at least one polyalkene-based supramolecular polymer is derived from the condensation of at least one polyalkene polymer functionalized with at least one reactive group, together with at least one graft functionalized with at least one reactive group capable of reacting with the at least one reactive group of the functionalized polyalkene polymer, wherein the at least one graft comprises at least one group capable of forming at least three H-bonds.

4. The composition according to claim 3, wherein the at least one functionalized polyalkene is chosen from compounds of formula (A):
HX—R—X′H (A)
wherein
XH and X′H are reactive groups, with X and X′, which may be identical or different, chosen from O, S, NH and NRₐ, wherein Rₐ represents a C₁-C₆ alkyl group;
R represents a homopolymer or a copolymer comprising at least one monounsaturated or polyunsaturated C₂-C₁₀ alkene.

5. The composition according to claim 3, wherein the at least one functionalized polyalkene is chosen from hydroxyl-terminated poly(ethylene-butylene)s, hydrogenated and nonhydrogenated hydroxyl-terminated polybutadienes, and hydrogenated and nonhydrogenated hydroxyl-terminated polyisoprenes.

6. The composition according to claim 3, wherein the at least one functionalized graft comprises at least one ureidopyrimidone group.

7. The composition according to claim 3, wherein the at least one reactive group of the at least one functionalized graft is an isocyanate group.

8. The composition according to claim 3, wherein the at least one functionalized graft is chosen from compounds of formula (B): 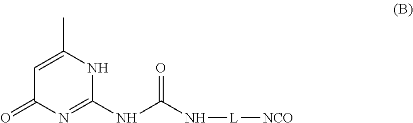
wherein
L is chosen from: phenylene; 1,4-nitrophenyl; 1,2-ethylene; 1,6-hexylene; 1,4-butylene; 1,6-(2,4,4-trimethylhexylene); 1,4-(4-methylpentylene); 1,5-(5-methylhexylene); 1,6-(6-methylheptylene); 1,5-(2,2,5-trimethylhexylene); 1,7-(3,7-dimethyloctylene); -isophorone-; 4,4′-methylenebiscyclohexylene; tolylene; 2-methyl-1,3-phenylene; 4-methyl-1,3-phenylene; and 4,4-biphenylenemethylene.

9. The composition according to claim 1, wherein the at least one polyalkene-based supramolecular polymer is obtained from the condensation of at least one polymer (A1) comprising a polyalkene part, wherein the polymer (A1) is functionalized with at least one reactive group (B1), together with at least one molecule (A3) comprising at least one reactive group (B2), wherein the at least one molecule (A3) is such that, after reaction of the (B1) and (B2) groups, an entity capable of forming at least three H-bonds, is formed.

10. The composition according to claim 9, wherein the at least one molecule (A3) is such that, after reaction of the (B1) and (B2) groups, an entity capable of forming at least four H-bonds is formed.

11. The composition according to claim 9, wherein the at least one polymer (A1) is chosen from compounds of formula (C1):
CON-L-NCO—X—R—X′—CON-L-NCO (C1)
wherein
X and X′, which may be identical or different, are chosen from O, S, NH and NRₐ, wherein Rₐ represents a C₁-C₆ alkyl group;
R represents a homopolymer or a copolymer comprising at least one monounsaturated or polyunsaturated C₂-C₁₀ alkene; and
L is chosen from: phenylene; 1,4-nitrophenyl; 1,2-ethylene; 1,6-hexylene; 1,4-butylene; 1,6-(2,4,4-trimethylhexylene); 1,4-(4-methylpentylene); 1,5-(5-methylhexylene); 1,6-(6-methylheptylene); 1,5-(2,2,5-trimethylhexylene); 1,7-(3,7-dimethyloctylene); -isophorone-; 4,4′-methylenebiscyclohexylene; tolylene; 2-methyl-1,3-phenylene; 4-methyl-1,3-phenylene; and 4,4-biphenylenemethylene.

12. The composition according to claim 9, wherein the at least one molecule (A3) is 6-methylisocytosine of formula:

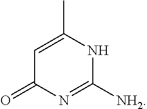

13. The composition according to claim 1, wherein the at least one polyalkene-based supramolecular polymer is chosen from compounds of formula C: 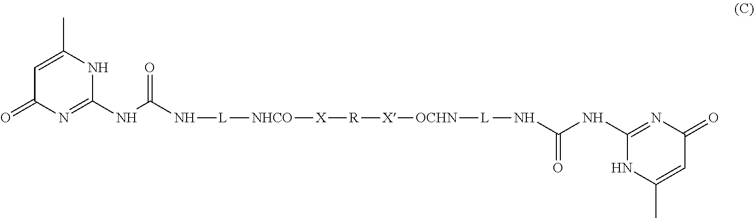
wherein
X and X′, which may be identical or different, are chosen from O, S, NH and NRₐ, wherein Rₐ represents a C₁-C₆ alkyl group;
R represents a homopolymer or a copolymer comprising at least one monounsaturated or polyunsaturated C₂-C₁₀ alkene; and
L is chosen from: phenylene; 1,4-nitrophenyl; 1,2-ethylene; 1,6-hexylene; 1,4-butylene; 1,6-(2,4,4-trimethylhexylene); 1,4-(4-methylpentylene); 1,5-(5-methylhexylene); 1,6-(6-methylheptylene); 1,5-(2,2,5-trimethylhexylene); 1,7-(3,7-dimethyloctylene); -isophorone-; 4,4′-methylenebiscyclohexylene; tolylene; 2-methyl-1,3-phenylene; 4-methyl-1,3-phenylene; and 4,4-biphenylenemethylene.

14. The composition according to claim 1, wherein
the at least one first monomer is chosen from:
methacrylates of formula CH₂═C(CH₃)—COOR1
in which R1 represents a linear or branched unsubstituted alkyl group comprising from 1 to 4 carbon atoms or a C₄ to C₁₂ cycloalkyl group,
acrylates of formula CH₂═CH—COOR2
in which R2 represents a C₄ to C₁₂ cycloalkyl group,
(meth)acrylamides of formula: 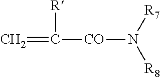
wherein R7 and R8, which may be identical or different, represent a hydrogen atom or a linear or branched C₁ to C₁₂ alkyl group, or R7 represents H and R8 represents a 1,1-dimethyl-3-oxobutyl group,
and R′ represents H or methyl, and
the at least one second monomer is chosen from:
acrylates of formula CH₂═CHCOOR3,
R3 represents an unsubstituted linear or branched C₁ to C₁₂ alkyl group, with the exception of the tert-butyl group, in which is optionally inserted at least one heteroatom chosen from O, N and S,
methacrylates of formula CH₂═C(CH₃)—COOR4,
R4 represents an unsubstituted linear or branched C₆ to C₁₂ alkyl group in which is optionally inserted at least one heteroatom chosen from O, N and S,
vinyl esters of formula R5-CO—O—CH═CH₂,
R5 represents a linear or branched C₄ to C₁₂ alkyl group;
C₄ to C₁₂ alkyl vinyl ethers, and
N—(C₄ to C₁₂ alkyl)acrylamides.

15. The composition according to claim 14, wherein the at least one second monomer is chosen from N-octylacrylamide.

16. The composition according to claim 1, wherein
the at least one first block comprises at least one acrylate monomer of formula CH₂═CH—COOR2 in which R2 represents a C₄ to C₁₂ cycloalkyl group and at least one methacrylate monomer of formula CH₂═C(CH₃)—COOR′2 in which R′2 represents a C₄ to C₁₂ cycloalkyl group; and
the at least one second block comprises at least one second monomer whose corresponding homopolymer has a glass transition temperature of less than or equal to 20° C. and at least one additional monomer.

17. The composition according to claim 16, wherein the at least one additional monomer is acrylic acid.

18. The composition according to claim 16, wherein R2 and R′2 represent, independently or simultaneously, an isobornyl group.

19. The composition according to claim 1, wherein the at least one block ethylenic copolymer comprises, from 50% to 80% by weight of isobornyl methacrylate/acrylate, from 10% to 30% by weight of isobutyl acrylate, and from 2% to 10% by weight of acrylic acid, relative to the total weight of the at least one block ethylenic copolymer.

20. The composition according to claim 1, wherein the at least one volatile solvent is chosen from water, non-silicone, and silicone organic solvents.

21. The composition according to claim 1, further comprising at least one additional silicone compound chosen from polysiloxanes having a viscosity of greater than 100 cst.

22. The composition according to claim 21, wherein the at least one polysiloxane is chosen from oils of polydimethylsiloxane type and silicone resins.

23. The composition according to claim 1, further comprising at least one pigment.

24. A process for treating keratin fibers, comprising
applying to the keratin fibers a treatment composition, and
optionally drying the keratin fibers at a temperature above 40° C.,
wherein the treatment composition comprises
at least one polyalkene-based supramolecular polymer,
at least one block ethylenic copolymer comprising
at least one first block having a glass transition temperature (Tg) of greater than or equal to 40° C. which comprises at least one first monomer, whose corresponding homopolymer has a glass transition temperature of greater than or equal to 40° C., and
at least one second block having a glass transition temperature of less than or equal to 20° C. which comprises at least one second monomer, whose corresponding homopolymer has a glass transition temperature of less than or equal to 20° C.,
wherein the at least one first block and the at least one second block are linked to one another via a random intermediate segment comprising at least one constituent monomer of the at least one first block and at least one constituent monomer of the at least one second block,
and wherein the at least one block copolymer has a polydispersity index I of greater than 2, and
at least one volatile solvent.
